(19) United States
(12) Patent Application Publication
(10) Pub. No.: US 2005/0204408 A1
(43) Pub. Date: Sep. 15, 2005

(54) Tropoelastin derivatives

(76) Inventor: Anthony S. Weiss, Sydney (AU)

Correspondence Address: NIKOLAI & MERSEREAU, P.A., 900 SECOND AVENUE SOUTH, SUITE 820, MINNEAPOLIS, MN, 55402, US

(21) Appl. No.: 11/053,710

(22) Filed: Feb. 8, 2005

Related U.S. Application Data
(63) Continuation of application No. 09/463,091, filed on Mar. 31, 2000.

(30) Foreign Application Priority Data
Jul. 17, 1998 (WO) PCT/AU98/00564

(51) Int. Cl. A01K 67/00; C12Q 1/68; C07H 21/04; C12N 9/66; C12N 15/09

(52) U.S. Cl. 800/8; 435/6; 435/69.1; 435/218; 435/320.1; 435/325; 536/23.2

(57) ABSTRACT

The invention relates to derivatives of tropoelastin and variants of those derivatives. The invention further provides expression products and hybrid molecules of the derivatives and variants of the invention. The invention further provides methods for the production of the derivatives, variants, expression products and hybrid molecules. Further provided are formulations, cross-linked structures and implants comprising the derivatives, variants, expression products and hybrid molecules of the invention. Further provided are uses of the derivatives, variants, expression products and hybrid molecules of the invention.

### TECHNICAL FIELD

The present invention relates to derivatives of human tropoelastin and variants thereof, to genetic constructs encoding the amino acid sequences of the derivatives and variants and to uses of the derivatives and variants. In particular, the derivatives of the present invention have elastin-like properties or macro-molecular binding properties.

### BACKGROUND ART

There are various forms of tropoelastin that typically appear to consist of two types of alternating domains: those rich in hydrophobic amino acids (responsible for the elastic properties) and those rich in lysine residues (responsible for cross-link formation). Hydrophobic and cross-linking domains are encoded in separate exons (Indik et al 1987).

The 26 A region of human tropoelastin is unique amongst tropoelastin domains in that, due to the absence of lysine, this region does not participate in elastin cross-link formation. Furthermore, this region is a serine-rich domain and lacks hydrophobic stretches, indicating that it is unlikely to contribute to the elasticity of tropoelastin. There is otherwise limited information on the structure and functional relationships of the 26 A region (Bedell-Hogan et al., 1993).

The gene for tropoelastin is believed to be present as a single copy in the mammalian genome, and is expressed in the form of multiple transcripts, distinguished by alternative splicing of the pre-mRNA (Indik et al, 1990; Oliver et al, 1987). Modest expression of a natural human tropoelastin sequence has been achieved by Indik et al (1990) using cDNA, providing free polypeptide which unfortunately was unstable.

Expression of substantial amounts of human tropoelastin using synthetic polynucleotides is reported in WO94/14958. In particular, a construct, SHEL, providing substantial amounts of full length human tropoelastin is described.

### DESCRIPTION OF THE INVENTION

In the specification and claims, “derivatives of human tropoelastin” or “tropoelastin derivatives” means novel peptides, polypeptides or proteins which contain amino acid sequences derived from the native amino acid sequences of human tropoelastin molecules. The amino acid sequences of the derivatives of human tropoelastin may be derived from any of the amino acid sequences of the isoforms of human tropoelastin. Derivatives of human tropoelastin are distinguished from human tropoelastin molecules in that the amino acid sequences of derivatives are altered with respect to native tropoelastin sequences by substitution, addition or deletion of residues, or a combination of these alterations, in derivative amino acid sequences.

In a first aspect, the present invention provides derivatives of human tropoelastin which have elastin-like properties. Elastin-like properties are a combination of elastic properties, including the phenomenon of recoil following molecular distention under appropriate conditions, and the ability to be cross-linked to other elastin molecules and/or other elastin-like molecules.

In a second aspect, the present invention provides derivatives of human tropoelastin which have macro-molecular binding properties including the ability to bind glycosaminoglycans.

In a third aspect, the present invention provides derivatives of human tropoelastin which have elastin-like properties and macro-molecular binding properties.

The present invention further provides amino acid sequence variants of the derivatives of the invention. In the specification and claims “variants” means amino acid sequences which retain the properties of the corresponding derivative of human tropoelastin, for example, elastin-like properties or macro-molecular binding properties, or a combination of elastin-like properties and macro-molecular binding properties, and have an amino acid sequence which is homologous with the amino acid sequence of the corresponding derivative. For the purposes of this description, “homology” between the amino acid sequence of a particular derivative of human tropoelastin and another amino acid sequence connotes a likeness short of identity, indicative of a derivation of one sequence from the other. In particular, an amino acid sequence is homologous to a derivative of human tropoelastin if the alignment of that amino acid sequence with the sequence of the derivative of human tropoelastin reveals a similarity of about 65% over any 20 amino acid stretch or over any repetitive element of the molecules shorter than 20 amino acids in length. Such a sequence comparison can be performed via known algorithims, such as that of Lipman and Pearson (1985). Similarity is observed between amino acids where those amino acids have a side chain which confers a similar chemical property in the same chemical environment. For example, threonine and serine are similar amino acids; aspartic acid and glutamic acid are similar amino acids; valine, leucine and isoleucine are similar amino acids etc. Thus, an amino acid sequence may be considered homologous with the amino acid sequence of a human tropoelastin derivative because the alignment of those sequences reveals a similarity of 65%, although at each amino acid position in the aligned sequences, none of the residues are identical.

Inasmuch as the present invention provides derivatives of human tropoelastin and amino acid sequence variants of those derivatives, the invention thus extends to amino acid sequence variants incorporating amino acid sequences of non-human tropoelastins. Amino acid sequence variants which are non-human tropoelastin derivatives, or are based all, or in part, on non-human tropoelastin derivatives retain properties of the corresponding derivative of non-human tropoelastin, for example, elastin-like properties or macro-molecular binding properties, or a combination of elastin-like properties and macro-molecular binding properties, and have an amino acid sequence which is homologous with the amino acid sequence of the corresponding human derivative. The variants of the invention also include variants of the non-human tropoelastin derivatives, or of derivatives based on the non-human tropoelastin derivatives. “Homology” between the amino acid sequence of a particular derivative of non-human tropoelastin and another amino acid sequence connotes a likeness short of identity, indicative of a derivation of one sequence from the other. In particular, an amino acid sequence is homologous to a derivative of non-human tropoelastin if the alignment of that amino acid sequence with the sequence of the derivative of non-human tropoelastin reveals a similarity of about 65% over any 20 amino acid stretch or over any repetitive element of the molecules shorter than 20 amino acids in length. The skilled addressee will understand that species that are substantially phylogenetically related to humans express tropoelastin molecules which consist of amino acid sequences with homology to human tropoelastin amino acid sequences. Indeed, amino acid sequences of non-human tropoelastins have been determined, including the amino acid sequences of chick tropoelastin, bovine tropoelastin and rat tropoelastin (Bressan et al. 1987, Raju et al. 1987, Pierce et al 1992) and over multiple regions, these are homologous with the human tropoelastin amino acid sequences. The skilled addressee will recognise therefore, that derivatives of human tropoelastin and amino acid sequence variants of those derivatives will necessarily encompass corresponding tropoelastin amino acid sequences from these and other non-human species.

The present invention provides a tropoelastin derivative comprising the amino acid sequence of SHELδmodified (SEQ ID NO:5). The amino acid sequence of SHELδmodified and the alignment of that amino acid sequence with the human tropoelastin sequence is shown in FIG. 5.

The invention also provides an amino acid sequence variant of the derivative comprising the amino acid sequence of SHELδmodified.

The invention also provides a polynucleotide encoding a tropoelastin derivative comprising the amino acid sequence of SHELδmodified. The nucleotide sequence encoding SHELδmodified is shown in FIG. 3 (SEQ ID NO: 4). Preferably the polynucleotide comprises the nucleotide sequence which corresponds to SHELδmodified shown in FIG. 3.

The invention also provides a polynucleotide encoding an amino acid sequence variant of the derivative SHELδmodified.

The present invention further provides a synthetic polynucleotide encoding a tropoelastin derivative comprising the amino acid sequence of SHELδ26A (SEQ ID NO:3). A synthetic polynucleotide is a molecule which comprises a nucleotide sequence that contains silent mutations with respect to the corresponding native polynucleotide molecule. The silent mutations enhance the expression of the synthetic polynucleotide. The amino acid sequence of SHELδ26A and the alignment of that amino acid sequence with the human tropoelastin sequence is shown in FIG. 2. The SHELδ26A derivative excludes the SHEL coding sequence corresponding to exon 26A. Preferably the synthetic polynucleotide comprises the sequence shown in FIG. 1 (SEQ ID NO:1) from nucleotide position 1 to 1676 contiguous with nucleotide position 1775 to 2210.

The invention also provides a polynucleotide encoding an amino acid sequence variant of the derivative SHELδ26A.

The invention also provides an amino acid sequence variant of the derivative comprising the amino acid sequence of SHELδ26A.

The present inventor has, for the first time, shown that the region encoded by exon 26A (peptide 26A) of the tropoelastin gene binds glycosaminoglycans (GAGs) (FIGS. 6A and B). GAGs are macro-molecules particularly associated with the extracellular environment. These molecules play an important role in the architecture and mechanical properties of connective tissues and mediate interactions with and availability of other molecules.

Thus, the present invention provides a tropoelastin derivative comprising the amino acid sequence of peptide 26A. Peptide 26A has the amino acid sequence: GADEGVRRSLSPELREGDPSSSQHLPSTPSSPRV (SEQ ID NO: 12) or GADEGVRRSLSPELREGDPSSSQHLPSTPSSPRF (SEQ ID NO: 13).

The present invention also provides an amino acid sequence variant of the derivative comprising the amino acid sequence of peptide 26A.

The invention also provides a polynucleotide encoding a tropoelastin derivative comprising the amino acid sequence of peptide 26A. Preferably the polynucleotide comprises the nucleotide sequence shown in FIG. 1 (SEQ ID NO: 1) from nucleotide position 1687 to 1778. Preferably the 3′ terminal codon is GTT (which encodes V) or TTT (which encodes F).

The invention also provides a polynucleotide encoding an amino acid sequence variant of the derivative comprising the amino acid sequence of peptide 26A.

In appreciating the GAG binding property of peptide 26A, the present inventor envisages the generation of novel subsets of hybrid molecules, comprising biological polymers which are linked to peptide 26A, wherein the peptide 26A imparts GAG binding activity to the polymer. In particular, the present inventor has recognised that the deletion or insertion of the peptide 26A amino acid sequence, or a variant of that amino acid sequence will alter GAG binding activity. Thus, the present invention relates to tropoelastin derivatives in which full length or partial length tropoelastin molecules have been modified by the addition of one or more exon 26A regions to enhance interactions with GAGs. Moreover, the invention relates to site directed modification of the amino acid sequence of peptide 26A so as to generate variants of the peptide 26A amino acid sequence which have altered affinity or altered specificity for GAGs. Tropoelastin derivatives or variants of the derivatives which contain altered GAG binding activity may be uncross-linked or cross-linked.

In another aspect, the invention provides a hybrid molecule. In the specification and claims, “hybrid molecule” means a molecule comprising a biological polymer which is linked to a tropoelastin derivative comprising the amino acid sequence of peptide 26A or an amino acid sequence variant of a derivative comprising the amino acid sequence of peptide 26A. Preferably the biological polymer is a protein. More preferably the protein is selected from the group consisting of growth factors, cytokines and antibodies. Alternatively the biological polymer is selected from the group consisting of lipids, sugars or nucleic acids.

In one embodiment, and where the biological polymer is a protein, the hybrid molecule is produced by recombinant DNA techniques, including for example the construction of a nucleotide sequence which encodes the biological polymer and the tropoelastin derivative comprising the amino acid sequence of peptide 26A, or the amino acid sequence variant of a derivative comprising the amino acid sequence of peptide 26 A, in a single open reading frame. Alternatively, the hybrid molecule may be produced synthetically by solid phase peptide synthesis, including, for example the methods of synthesis disclosed in Merrifield (1963) or Knorr et al. (1989). Examples of peptide synthesis also include the synthesis methods used by peptide synthesisers of Perkin Elmer/Applied Biosystems, CA, US.

In another aspect, the invention provides a polynucleotide sequence encoding a hybrid molecule of the invention.

In another aspect, the invention provides a hybrid molecule which comprises a synthetic polymer which is linked in a tropoelastin derivative comprising the amino acid sequence of peptide 26A, or an amino acid sequence variant of the derivative comprising the amino acid sequence of peptide 26A.

The invention further provides a method of imparting or enhancing GAG binding activity to a biological polymer comprising the step of linking a tropoelastin derivative comprising the amino acid sequence of peptide 26A, or an amino acid sequence variant of peptide 26A with the biological polymer. Preferably the biological polymer is a protein.

The invention further provides a method of deleting or reducing GAG binding activity from a biological polymer comprising the step of deleting a tropoelastin derivative comprising the amino acid sequence of peptide 26A, or an amino acid sequence variant of peptide 26A from the biological polymer. Preferably the biological polymer is a protein.

The present invention also provides a tropoelastin derivative comprising the amino acid sequence of SHELgamma. SHELgamma has the amino acid sequence:

(SEQ ID NO: 9)

SAMGALVGLGVPGLGVGAGVPCFGAGADEGVRRSLSPELREGDPSSSQHL
PSTPSSPRVPGALAAAKAAKYGAAVPGVLGGLGALGGVGIPGGVVGAGPA
AAAAAAKAAAKAAQFGLVGAAGLGGLGVGGLGVPGVGGLGGIPPAAAAKA
AKYGAAGLGGVLGGAGQFPLGGVAARPGFGLSPIFPGGACLGKACG
RKRK.

The invention also provides an amino acid sequence variant of the derivative comprising the amino acid sequence of SHELgamma.

The invention also provides a polynucleotide encoding a tropoelastin derivative, the derivative comprising the amino acid sequence of SHELgamma. The nucleotide sequence of the polynucleotide SHELgamma (SEQ ID NO: 8) is shown in FIG. 8. In this nucleotide sequence, the first 9 codons from nucleotide position 948 to 974 are derived from the glutathione S-transferase (GST) fusion nucleotide sequence. Preferably the polynucleotide comprises the nucleotide sequence shown in FIG. 8. More preferably the polynucleotide comprises the nucleotide sequence shown in FIG. 8 from nucleotide sequence position 975 to 1547.

The invention also provides a polynucleotide encoding an amino acid sequence variant of the derivative comprising the amino acid sequence of SHELgamma.

The present invention also provides a polynucleotide encoding a tropoelastin derivative, the derivative comprising the amino acid sequence of SHELgamma excluding exon 26A. The nucleotide sequence of the polynucleotide SHELgamma excluding exon 26A (SEQ ID NO: 6) is shown in FIG. 7. In this nucleotide sequence, the first 5 codons from nucleotide position 948 to 962 are derived from the GST nucleotide sequence. SHELgamma excluding exon 26A has the following amino acid sequence:

(SEQ ID NO: 7)

VPGALAAAKAAKYGAAVPGVLGGLGALGGVGIPGGVVGAGPAAAAAAAKA
AAKAAQFGLVGAAGLGGLGVGGLGVPGVGGLGGIPPAAAAKAAKYGAAGL
GGVLGGAGQFPLGGVAARPGFGLSPIFPGGACLGKACGRKRK.

Preferably the polynucleotide comprises the nucleotide sequence shown in SEQ ID NO:6. More preferably the polynucleotide comprises the nucleotide sequence shown in SEQ ID NO: 6 from nucleotide sequence position 15 to 441.

The invention also provides a polynucleotide encoding an amino acid sequence variant of the derivative comprising the amino acid sequence of SHELgamma excluding exon 26A.

The invention also provides a tropoelastin derivative comprising the amino acid sequence of SHELgamma excluding exon 26A.

The invention also provides an amino acid sequence variant of the derivative comprising SHELgamma excluding exon 26A.

The derivatives of the invention based on SHELgamma can also be produced by in vitro biochemical cleavage of tropoelastin products such as SHEL, so as to release a carboxy-terminal fragment. The carboxy-terminal fragment may be purified by reverse phase HPLC.

The present invention also provides a tropoelastin derivative comprising the amino acid sequence of SHEL31-36. SHEL31-36 has the following amino acid sequence:

(SEQ ID NO: 10)

GIPPAAAAKAAKYGAAGLGGVLGGAGQFPLGGVAARPGFGLSPIFPGGAC
LGKACGRKRK.

SHEL31-36 retains a crosslinking domain. As a consequence of its elastin-like properties, it is envisaged that this and related tropoelastin derivatives can be used to interfere with tropoelastin deposition and formation of unaltered elastic fibre.

The invention also provides an amino acid sequence variant of the derivative comprising the amino acid sequence of SHEL31-36.

The invention also provides a polynucleotide encoding a tropoelastin derivative, the derivative comprising the amino acid sequence of SHEL31-36. Preferably the polynucleotide comprises the nucleotide sequence shown in FIG. 1 (SEQ ID NO:1) from nucleotide position 2022 to 2210.

The invention also provides a polynucleotide encoding an amino acid variant of the derivative comprising the amino acid sequence of SHEL31-36.

The present invention also provides a tropoelastin derivative, comprising the amino acid sequence of SHEL32-36. SHEL32-36 has the following amino acid sequence:

(SEQ ID NO: 11)

GAAGLGGVLGGAGQFPLGGVAARPGFGLSPIFPGGACLGKACGRKRK.

The invention also provides an amino acid sequence variant of the derivative comprising the amino acid sequence of SHEL32-36.

The invention also provides a polynucleotide encoding a tropoelastin derivative, the derivative comprising the amino acid sequence of SHEL32-36. Preferably the polynucleotide comprises the nucleotide sequence shown in FIG. 1 (SEQ ID NO: 1) from nucleotide position 2061 to 2210.

The present invention also provides a polynucleotide encoding an amino acid sequence variant of the derivative comprising the amino acid sequence of SHEL32-36.

As a consequence of its elastin-like properties, it is envisaged that SHEL32-36 and related tropoelastin derivatives can be used to interfere with tropoelastin deposition and formation of an unaltered elastic fibre.

The present invention also provides a tropoelastin derivative, comprising the amino acid sequence of SHEL26-36. SHEL26-36 has the following amino acid sequence:

(SEQ ID NO: 14)

AAAGLGAGIPGLGVGVGVPGLGVGAGVPGLGVGAGVPGFGAGADEGVRRS
LSPELREGDPSSSQHLPSTPSSPRVPGALAAAKAAKYGAAVPGVLGGLGA
LGGVGIPGGVVGAGPAAAAAAAKAAAKAAQFGLVGAAGLGGLGVGGLGVP
GVGGLGGIPPAAAAKAAKYGAAGLGGVLGGAGQFPLGGVAARPGFGLSPI
FPGGACLGKACGRKRK

The invention also provides an amino acid sequence variant of the derivative comprising the amino acid sequence of SHEL26-36.

The invention also provides a polynucleotide encoding a tropoelastin derivative, the derivative comprising the amino acid sequence of SHEL26-36. Preferably the polynucleotide comprises the nucleotide sequence shown in FIG. 1 from nucleotide position 1554-2210.

The present invention also provides a tropoelastin derivative, comprising the amino acid sequence of SHEL26-36 excluding exon 26A. SHEL26-36 excluding exon 26A has the following amino acid sequence:

(SEQ ID NO: 15)

AAAGLGAGIPGLGVGVGVPGLGVGAGVPGLGVGAGVPGFGAVPGALAAAK
AAKYGAAVPGVLGGLGALGGVGIPGGVVGAGPAAAAAAAKAAAKAAQFGL
VGAAGLGGLGVGGLGVPGVGGLGGIPPAAAAKAAKYGAAGLGGVLGGAGQ
FPLGGVAARPGFGLSPIFPGGACLGKACGRKRK

The invention also provides an amino acid sequence variant of the derivative comprising the amino acid sequence of SHEL26-36 excluding exon 26A.

The invention also provides a polynucleotide encoding a tropoelastin derivative, the derivative comprising the amino acid sequence of SHEL26-36 excluding exon 26A. Preferably the polynucleotide comprises the nucleotide sequence shown in FIG. 1 from nucleotide position 1554 to 1676 contiguous with 1776 to 2210.

The present invention also provides a polynucleotide encoding an amino acid sequence variant of the derivative comprising the amino acid sequence of SHEL26-36.

In another aspect the present invention provides a formulation comprising a tropoelastin derivative, a variant of the derivative or a hybrid molecule of the invention, together with a carrier or diluent.

Formulations of the derivatives, variants or hybrid molecules of the invention can be prepared in accordance with standard techniques appropriate to the field in which they are to be used.

The polynucleotides and synthetic polynucleotides of the invention can be provided in association with other polynucleotide sequences including 5′ and 3′ untranslated sequences, and 5′ upstream and 3′ downstream transcriptional regulatory sequences. The polynucleotides and synthetic polynucleotides may be provided as a recombinant DNA molecule including plasmid DNA.

The polynucleotides and synthetic polynucleotides of the invention can be prepared using the techniques of chemical synthesis or recombinant DNA technology, or by a combination of both techniques.

In a further aspect the invention provides a vector comprising a polynucleotide or synthetic polynucleotide encoding a tropoelastin derivative, a variant of the derivative or a hybrid molecule of the invention.

Vectors useful in this invention include plasmids, phages and phagemids. The polynucleotides and synthetic polynucleotides of the present invention can also be used in integrative expression systems or lytic or comparable expression systems.

Suitable vectors will generally contain origins of replication and control sequences which are derived from species compatible with the intended expression host. Typically these vectors include a promoter located upstream from the polynucleotide, together with a ribosome binding site if intended for prokaryotic expression, and a phenotypic selection gene such as one conferring antibiotic resistance or supplying an auxotrophic requirement. For production vectors, vectors which provide for enhanced stability through partitioning may be chosen. Where integrative vectors are used it is not necessary for the vector to have an origin of replication. Lytic and other comparable expression systems do not need to have those functions required for maintenance of vectors in hosts.

For E. coli typical vectors include pBR322, pBluescript II SK⁺, pGEX-2T, pTrc99A, pET series vectors, particularly pET3d, (Studier et al., 1990) and derivatives of these vectors. Derivatives include those plasmids with a modified protease recognition sequence to facilitate purification of a protein domain.

In another aspect the invention provides a cell capable of expressing a polynucleotide or a synthetic polynucleotide which encodes a derivative or variant of the invention, or a polynucleotide which encodes a hybrid molecule of the invention.

A preferred expression system is an E. coli expression system. However, the invention includes within its scope the use of other hosts capable of expressing protein from the polynucleotides designed for use in E. coli. The invention also includes the use of polynucleotides and synthetic polynucleotides suitable for use in other expression systems such as other microbial expression systems. These other expression systems include yeast, and bacterial expression systems, insect cell expression systems, and expression systems involving other eukaryotic cell lines or whole organisms.

Examples of E. coli hosts include E. coli B strain derivatives (Studier et al, 1990), and K-strain derivatives such as NM522 (Gough and Murray, 1983) and XL1-Blue (Bullock et al, 1987).

In a further aspect the present invention provides an expression product. In the specification and claims, “expression product” means a derivative or variant of the invention expressed by a cell containing a polynucleotide or a synthetic polynucleotide encoding a derivative or variant of the invention.

The expression products of the invention may be fused expression products which include all or part of a protein encoded by the vector in peptide linkage with the derivative or variant. They may also include, for example, an N-terminal methionine or other additional residues which do not permanently impair the elastin-like, or macro-molecular binding properties of the product.

Typically the fusion is to the N-terminus of the expression product. An example of a suitable protein is to the C-terminus of glutathione S-transferase. The fused protein sequence may be chosen in order to cause the expression product to be secreted or expressed as a cell surface protein to simplify purification or expressed as a cytoplasmic protein.

The expressed fusion products may subsequently be treated to remove the fused protein sequences to provide free tropoelastin derivative or variant. Treatment is typically through protease treatment or, in the case of secretion, removal is effected by endogenous host secretion machinery. An example of this is secretion by yeasts.

Non-fused systems include the introduction of or use of a pre-existing methionine codon. An example of this is the use of pET3a or pET3d in E. coli.

In another aspect the invention provides a polynucleotide encoding an expression product of the invention.

In another aspect the present invention provides a formulation comprising an expression product of the invention together with a carrier or diluent. The formulation of the expression product can be prepared in accordance with standard techniques appropriate to the field in which they are to be used.

According to a further aspect of the present invention there is provided a method for producing a tropoelastin derivative or a variant of the derivative comprising providing a vector containing a polynucleotide or a synthetic polynucleotide encoding the derivative or variant; introducing the vector into a suitable host cell; maintaining the cell in conditions suitable for expression of the polynucleotide or synthetic polynucleotide and isolating the derivative or variant of the invention. The method can be applied to the production of the expression products and hybrid molecules (in which the hybrid molecules comprise the peptide 26A or a variant thereof and a further amino acid sequence) of the invention, by providing a vector containing a polynucleotide encoding an expression product or a hybrid molecule; introducing the vector into a suitable host cell; maintaining the cell in conditions suitable for expression of the polynucleotide and isolating the expression product or hybrid molecule.

In one embodiment, the polynucleotide or synthetic polynucleotide encoding the derivative, variant, expression product or hybrid molecule of the invention is expressed in a host cell which is maintained in culture in vitro.

Alternatively, the polynucleotide or synthetic polynucleotide encoding the derivative, variant, expression product or hybrid molecule of the invention is expressed in a host cell which is maintained in vivo. Thus, in another embodiment, the polynucleotide or synthetic polynucleotide encoding the derivative, variant, expression product or hybrid molecule of the invention is expressed in a transgenic animal. Methods for the generation of transgenic animals are known in the art. Exemplary methods are described in Slack et al. 1991 and Janne et al. 1992.

The tropoelastin derivatives, variants of the derivatives, and hybrid molecules (in which the hybrid molecules comprise the peptide 26A or a variant thereof and a further amino acid sequence) of the invention may be produced by solid phase peptide synthesis, including, for example, the methods of synthesis disclosed in Merrifield (1963) or Knorr et al (1989). Examples of peptide synthesis also include the synthesis methods used by peptide synthesisers of Perkin Elmer/Applied Biosystems, CA, US. As an alternative to cell synthesis from a polynucleotide or synthetic polynucleotide, the expression products of the invention may be produced by solid phase peptide synthesis.

In a further aspect the present invention provides an implant formed from at least one tropoelastin derivative and/or variant of the derivative of the invention. The implant may alternatively contain at least one expression product and/or at least one hybrid molecule of the invention.

The implants are formed into the required shape by cross-linking the tropoelastin derivative, variant of the derivative, expression product, or hybrid molecule of the invention, in a mould which conforms to the desired shape of the implant. Where the implant is required to be used in sheet form the tropoelastin derivative, variant of the derivative, expression product, or hybrid molecule of the invention can be cross-linked on a flat surface. Relevant methodologies are described in, for example, U.S. Pat. No. 4,474,851 and U.S. Pat. No. 5,250,516. The elastomeric materials may be exclusively prepared from one or more tropoelastin derivatives, variants of the derivative, expression products, or hybrid molecules of the invention or may be composites prepared from one or more of these constituents together with other materials.

The tropoelastin derivatives or variants of the derivatives can be cross-linked to form elastin or elastin-like material or can be cross-linked in conjunction with other biological or synthetic molecules to form a composite material.

Thus in another aspect the invention provides a cross-linked complex which comprises at least one tropoelastin derivative of the invention and/or at least one variant of a derivative of the invention. The cross-linked complexes may additionally contain at least one expression product and/or at least one hybrid molecule of the invention, which may be cross-linked to the at least one tropoelastin derivative and/or variant of the derivative of the invention.

The cross-linking of the tropoelastin derivatives, variants of the derivatives, hybrid molecules and expression products of the invention can be achieved by chemical oxidation of lysine side chains using processes such as ruthenium tetroxide mediated oxidation and quinone mediated oxidation, or by using homobifunctional chemical cross-linking agents such as dithiobis (succinimidylpropionate), dimethyl adipimidate or dimethyl pimelimidate. Glutaraldehyde cross-linking is an important addition to this repetoire. Another alternative is the cross-linking of lysine and glutamic side chains.

The tropoelastin derivatives, variants of the derivatives, hybrid molecules and expression products of the invention may also be enzymatically cross-linked by methods including lysyl oxidase mediated oxidation or may be cross-linked using gamma irradiation.

### BRIEF DESCRIPTION OF THE DRAWINGS

FIG. 1: Nucleotide (SEQ ID NO: 1) and predicted amino acid (SEQ ID NO:2) sequences of synthetic human tropoelastin SHEL. The upper (numbered) nucleotide sequence represents the coding strand.

FIG. 2: Alignment of SHEL (SEQ ID NO:2)(upper line) and SHELδ26A (SEQ ID NO: 3) amino acid sequences.

FIG. 3: Nucleotide (SEQ ID NO: 4) and predicted amino acid (SEQ ID NO: 5) sequences of SHELδmodified.

FIG. 4: Alignment of SHELδmodified (SEQ ID NO: 4) (upper line) and SHEL (SEQ ID NO:1) nucleotide sequences.

FIG. 5: Alignment of SHELδmodified (SEQ ID NO: 5)(lower line) and SHEL (SEQ ID NO: 1) amino acid sequences.

FIG. 6A: HPLC elution profile of GST-exon 26A fusion protein tropoelastin derivative loaded in from heparin sepharose. 6B: Binding of peptide 26A (SEQ ID NO: 12 and SEQ ID NO: 13) to glycosaminoglycans.

FIG. 7: Nucleotide (SEQ ID NO: 6) and predicted amino acid (SEQ ID NO: 7) sequences of SHELgamma excluding exon 26A.

FIG. 8: Nucleotide (SEQ ID NO: 8) and predicted amino acid (SEQ ID NO: 9) sequences of SHELgamma.

### BEST METHOD OF PERFORMING THE INVENTION

The recombinant and synthetic procedures used for the synthesis of the derivatives, variants, expression products and hybrid molecules of the invention are described in standard texts such as Sambrook et al (1989).

Tropoelastin nucleotide sequences may be modified so as to provide derivatives, variants, expression products or hybrid molecules, by conventional site-directed or random mutagenesis. The sequences may also be modified by oligonucleotide-directed mutagenesis, which comprises the following steps:
1. synthesis of an oligonucleotide with a sequence that contains the desired nucleotide substitution (mutation);
2. hybridising the oligonucleotide to a template comprising a structural sequence encoding tropoelastin; and
3. using a DNA polymerase to extend the oligonucleotide as a primer.

Another approach which is particularly suited to situations where a synthetic polynucleotide encoding the tropoelastin derivative is prepared from oligonucleotide blocks bounded by restriction sites, is cassette mutagenesis where entire restriction fragments are replaced.

Purification of the derivatives, variants, expression products or hybrid molecules of the invention is performed using standard techniques including HPLC. The actual sequence of steps in the purification of a particular derivative, variant, expression product or hybrid molecule is limited by the environment from which the molecule is to be purified. By way of example, reference is made to the purification scheme disclosed in WO94/14958.

Formulations in accordance with the invention are formulated in accordance with standard techniques.

The amount of derivative, variant, expression product or hybrid molecule that may be combined with a carrier or diluent to produce a single dosage will vary depending on the situation in which the formulation is to be used and the particular mode of administration.

It will be understood also that specific doses for any particular host may be influenced by factors such as the age, sex, weight and general health of the host as well as the particular characteristics of the derivative, variant, expression product or hybrid molecule of the invention being used, and how it is administered.

Injectable preparations, for example, sterile injectable aqueous or oleagenous suspensions may be formulated according to the known art using suitable dispersing or wetting agents and suspending agents. The sterile injectable preparation may also be a sterile injectable solution or suspension in a non-toxic parenterally acceptable diluent or solvent. Among the acceptable vehicles or solvents that may be employed are water, Ringer's solution, alcohols and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid and organic solvents find use in the preparation of injectables.

Routes of administration, dosages to be administered as well as frequency of administration are all factors which can be optimised using ordinary skill in the art.

In addition, the derivatives, variants, expression products and hybrid molecules of the invention may be prepared as topical preparations for instance as anti-wrinkle and hand lotions using standard techniques for the preparation of such formulations. They may be prepared in aerosol form for, for instance, administration to a patient's lungs, or in the form of surgical implants, foods or industrial products by standard techniques.

SHEL

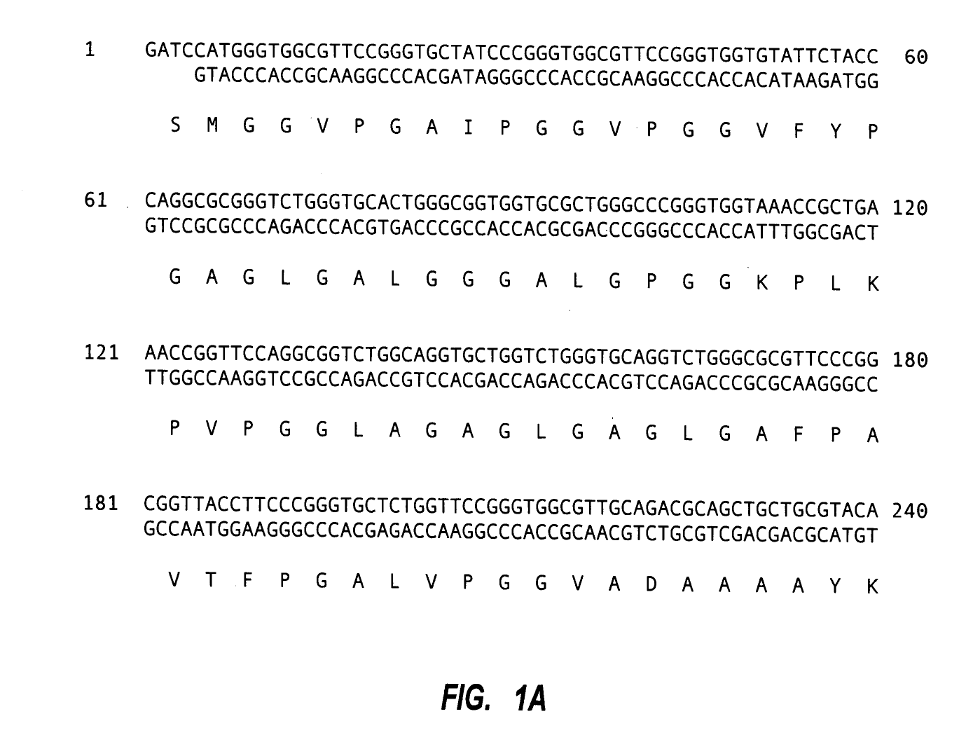

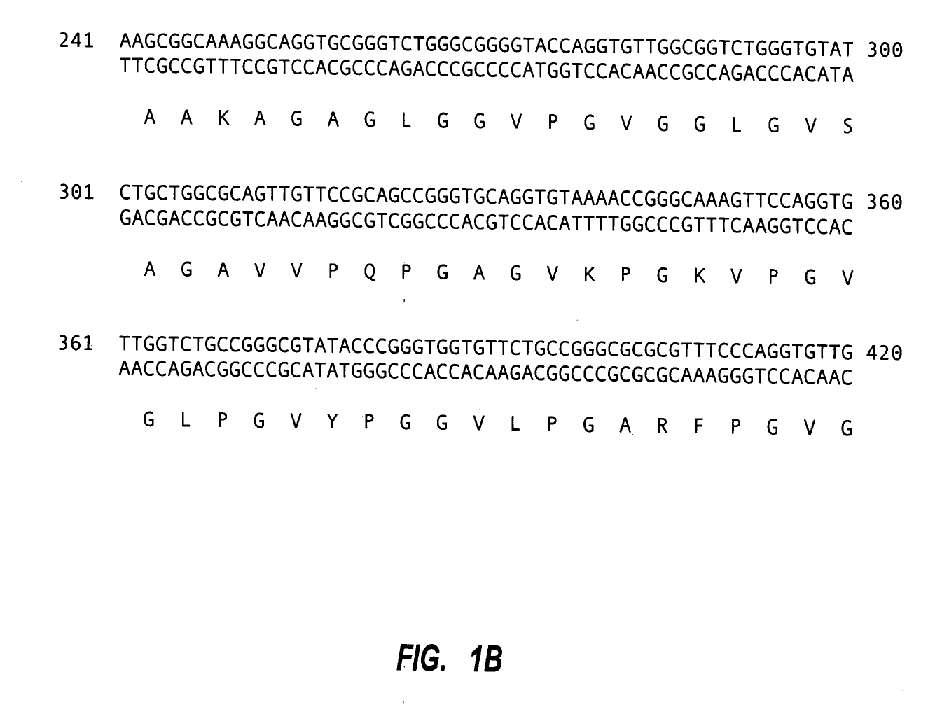

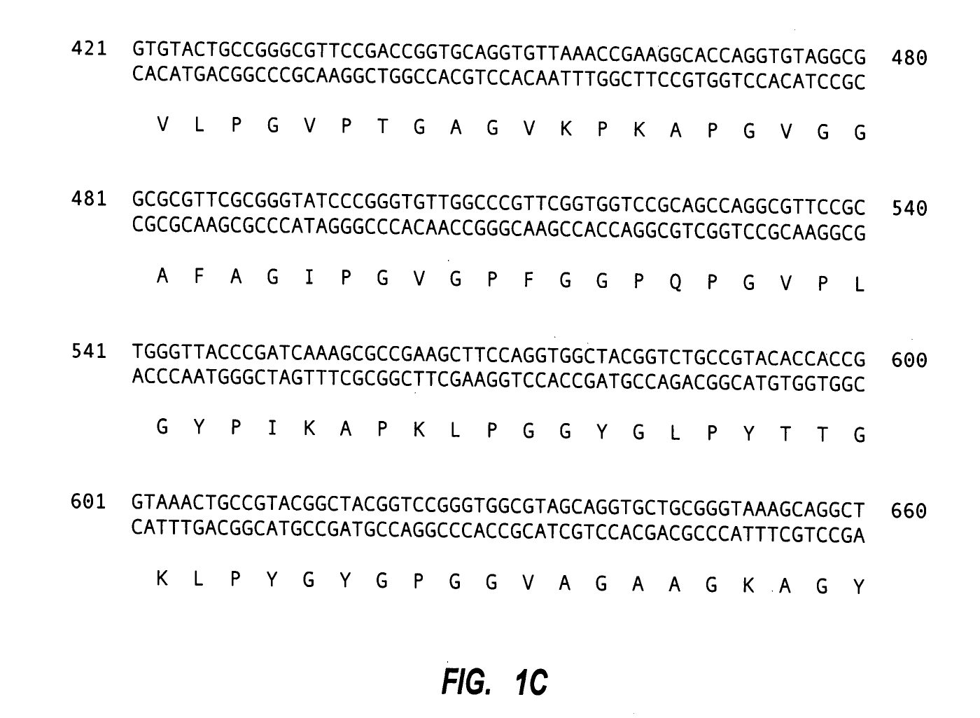

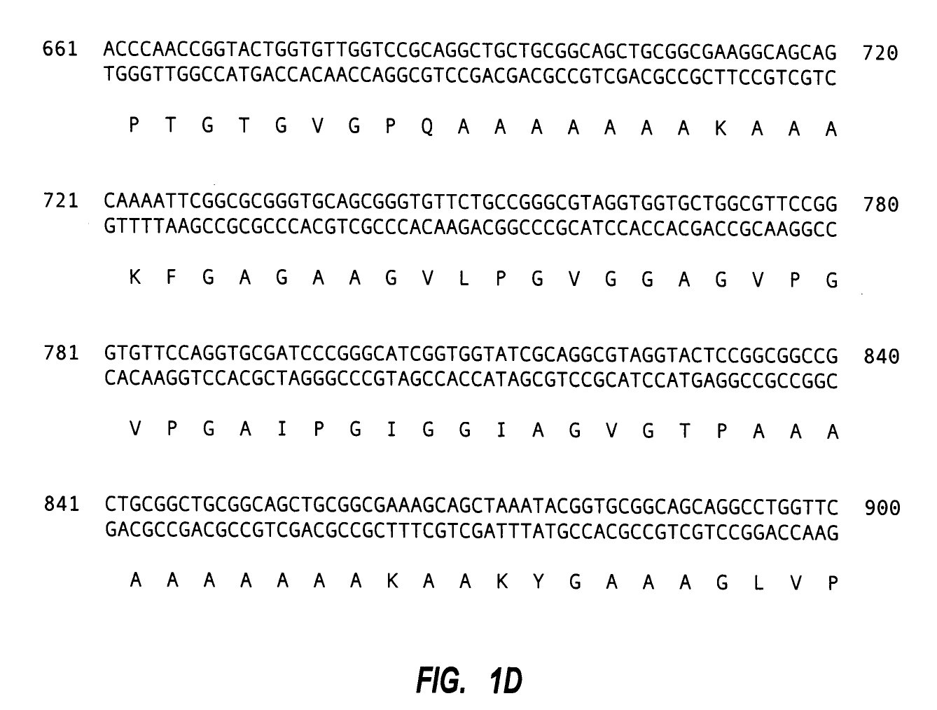

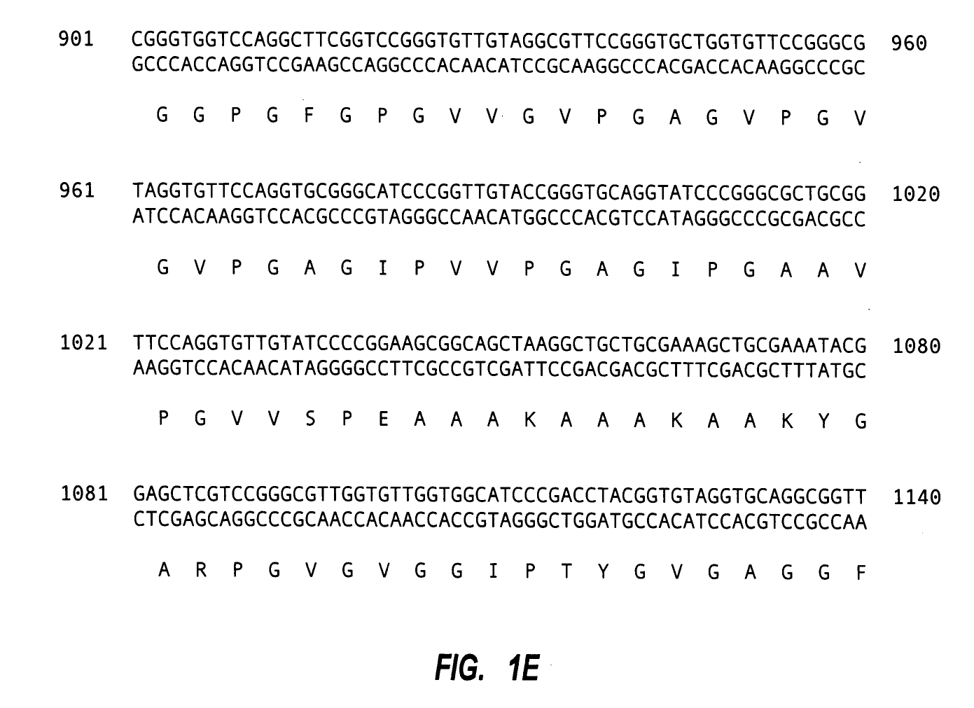

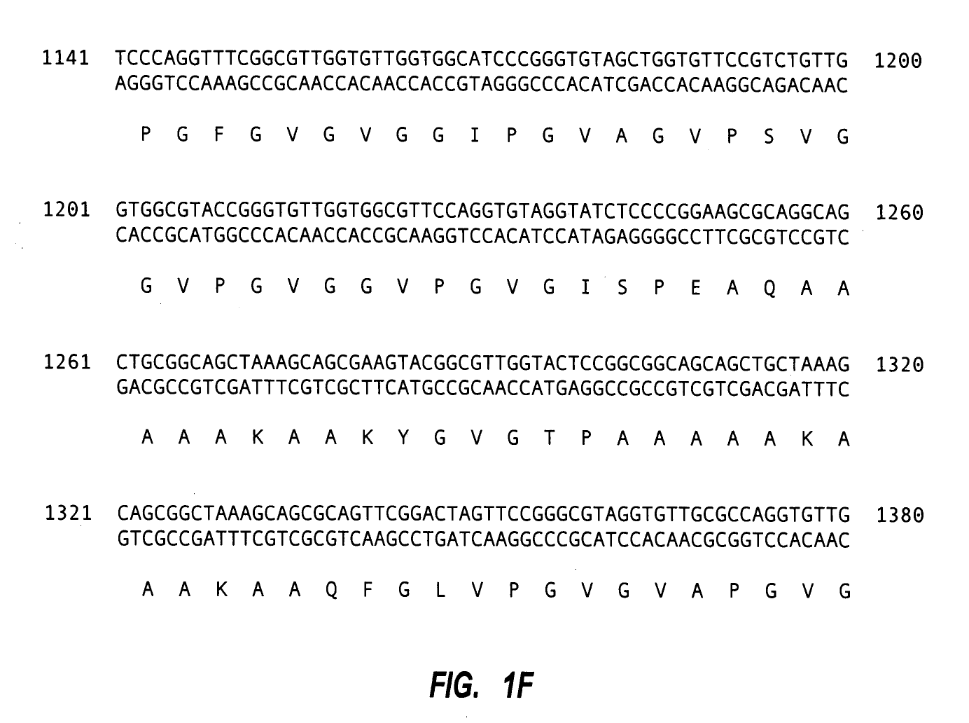

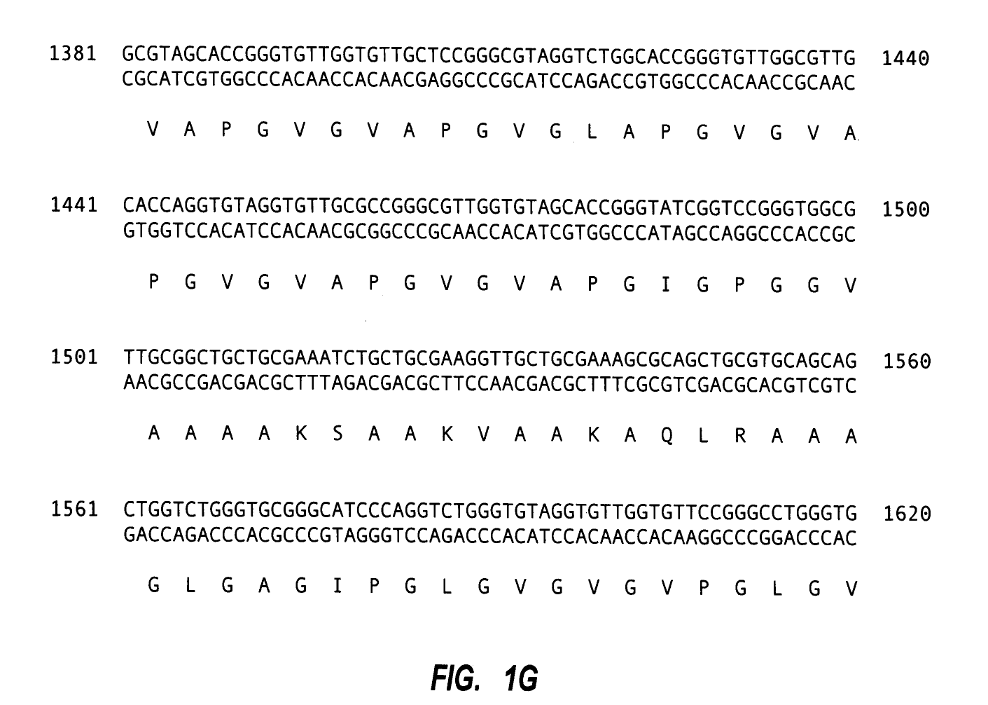

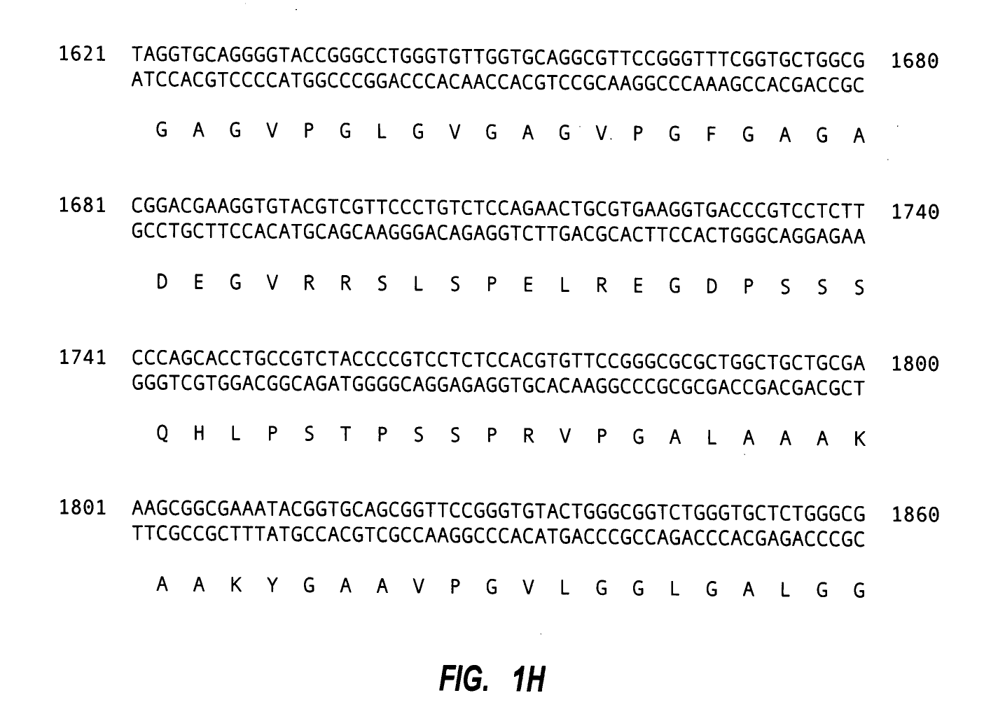

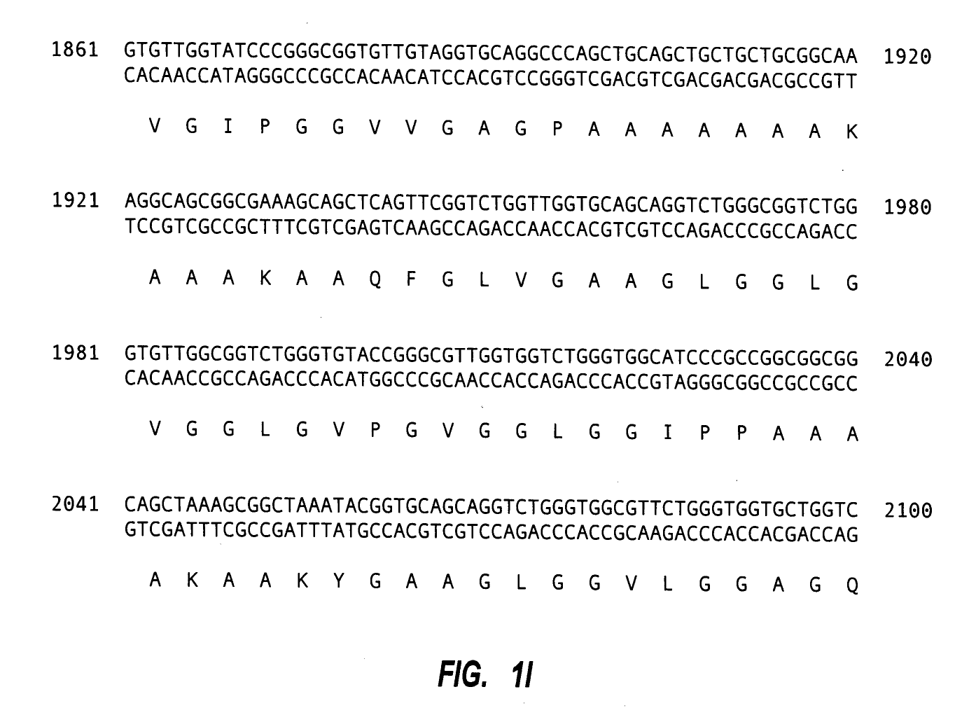

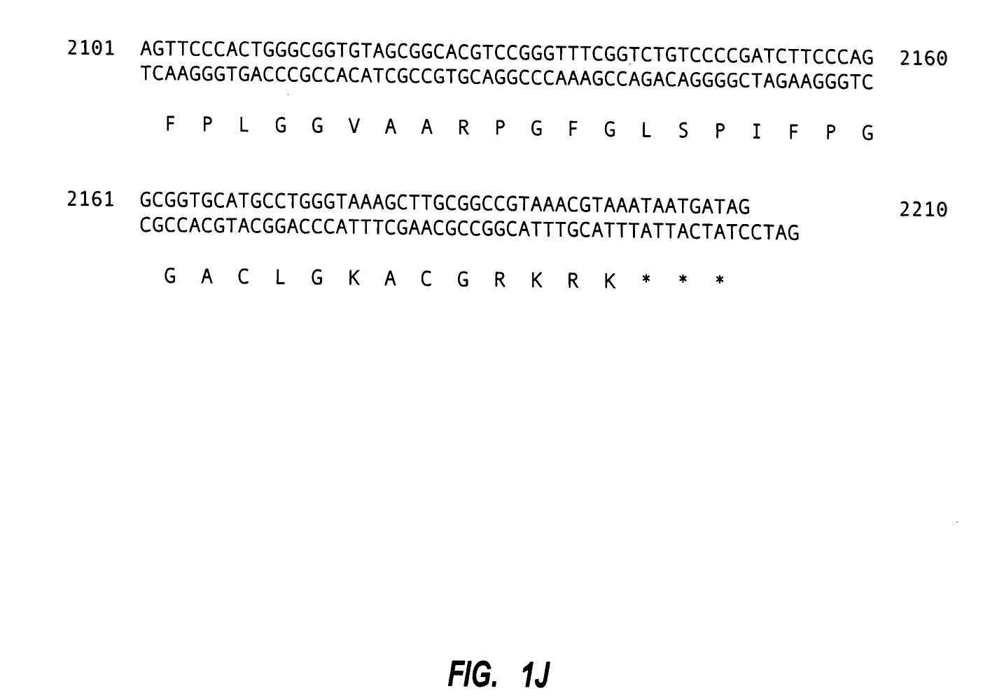

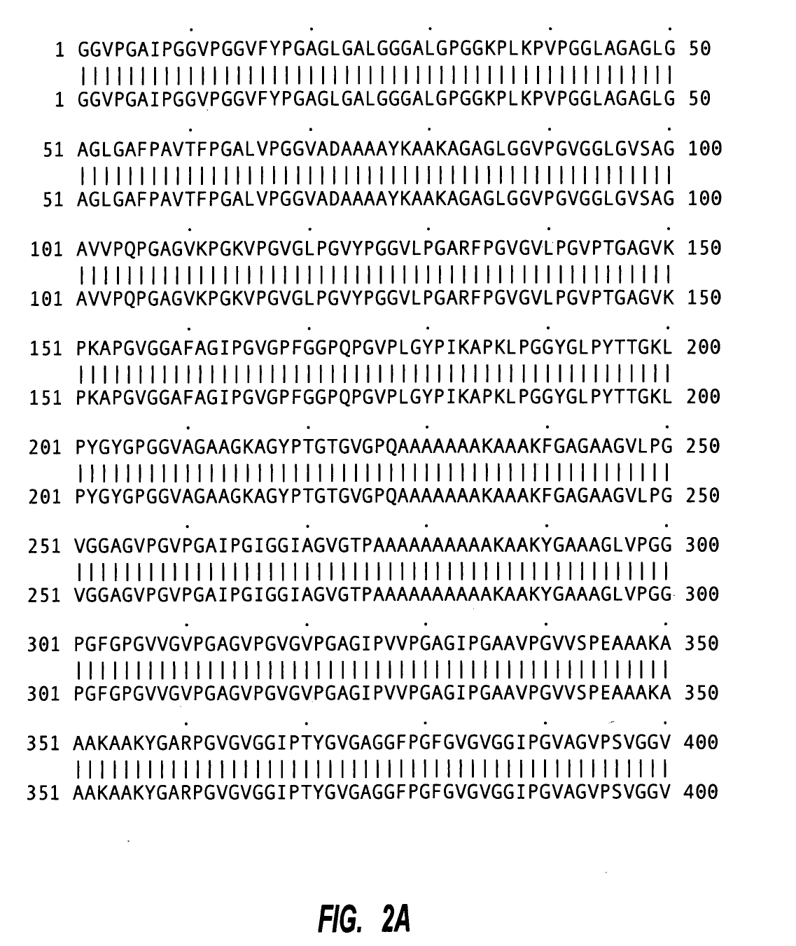

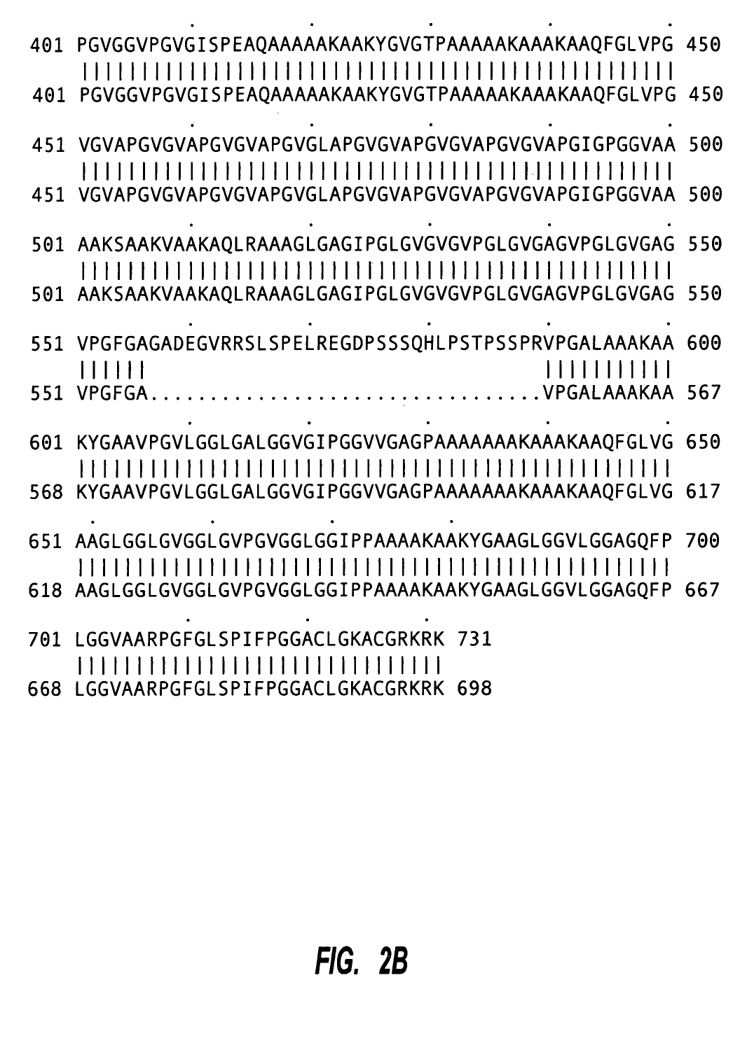

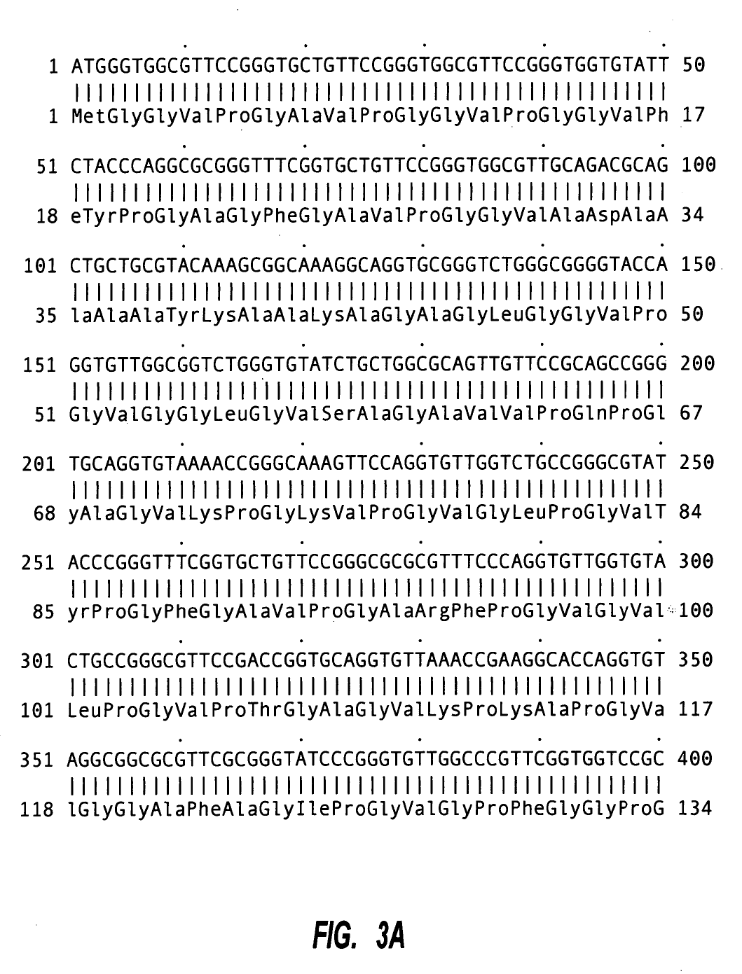

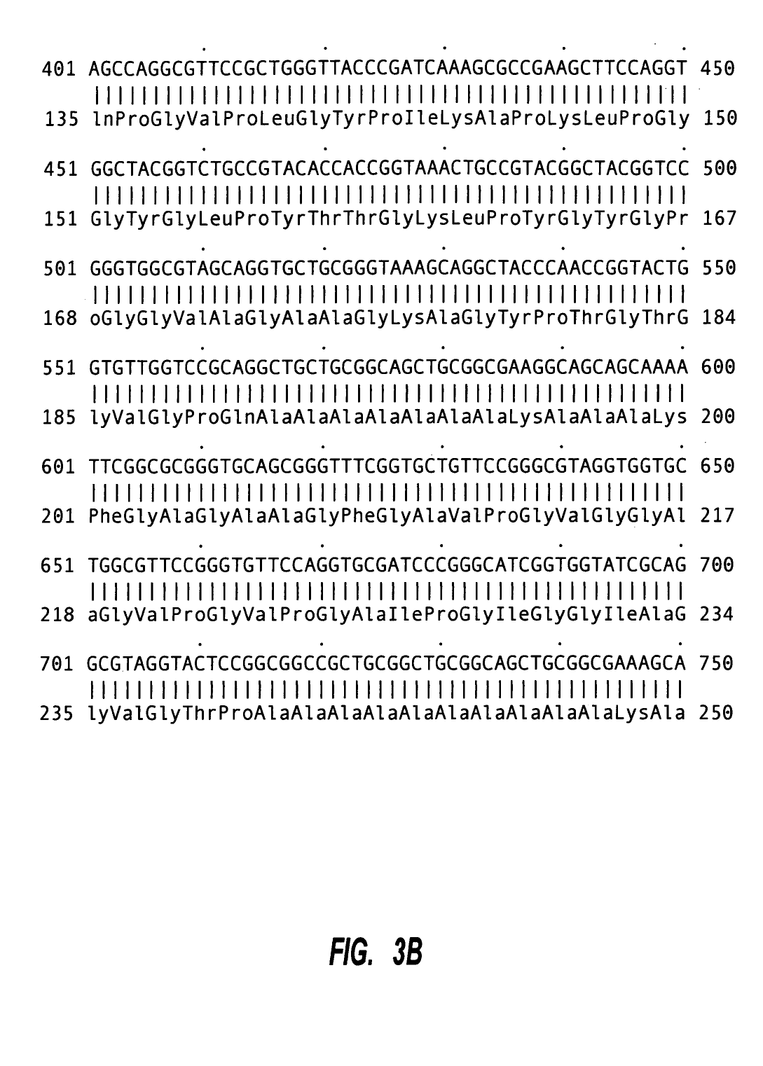

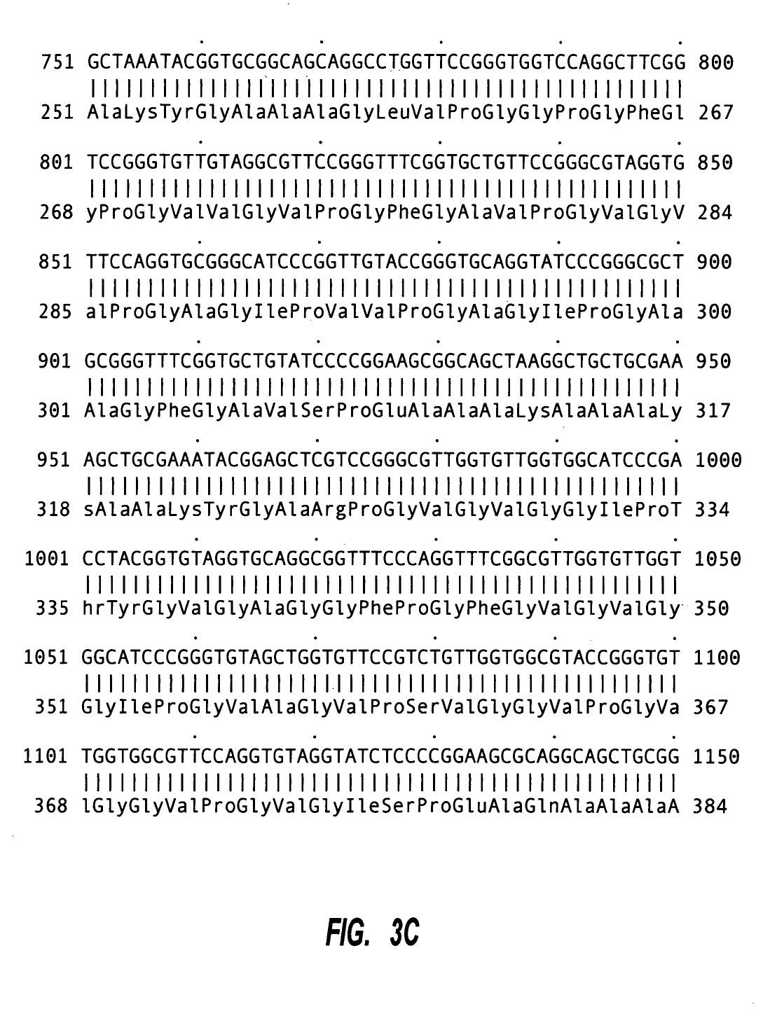

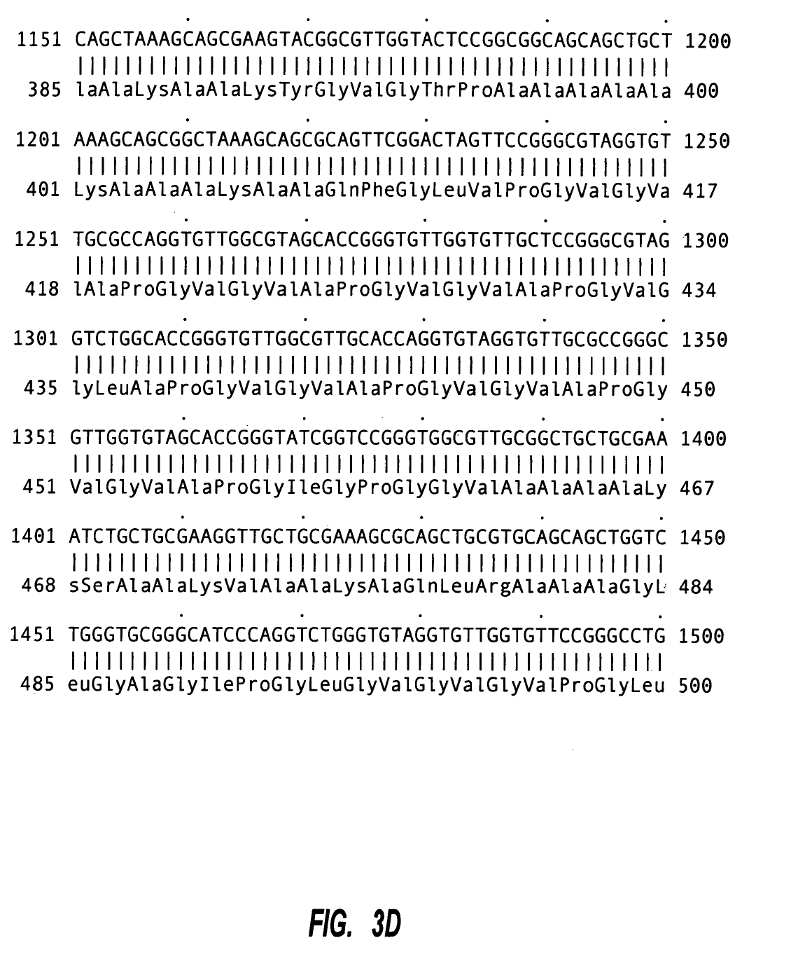

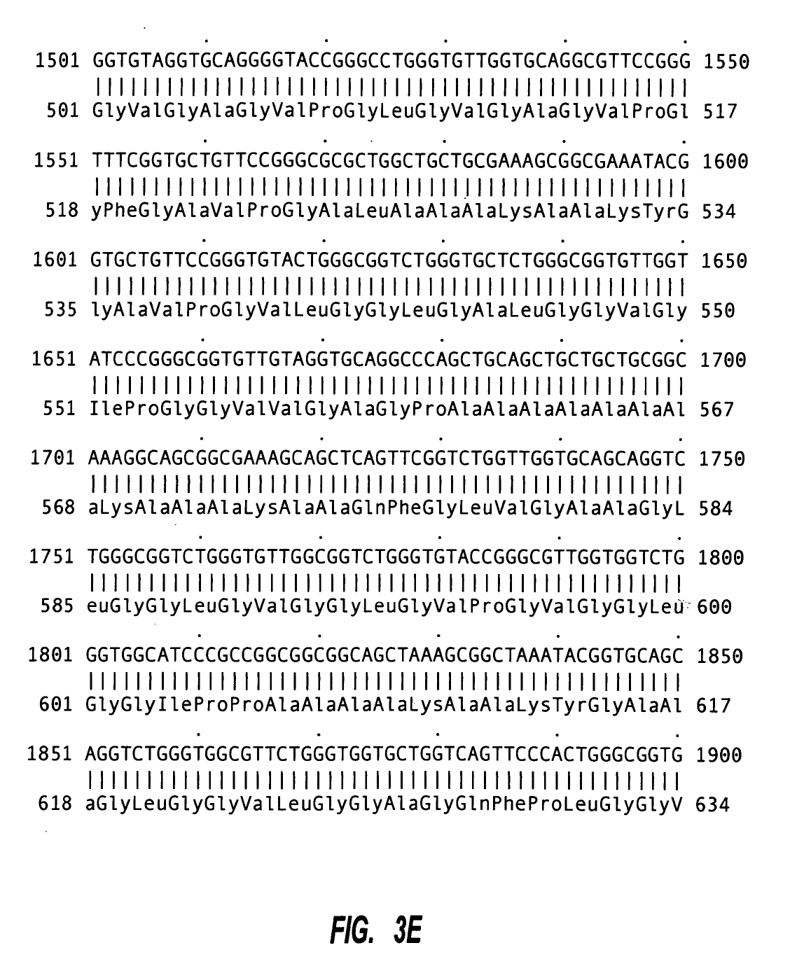

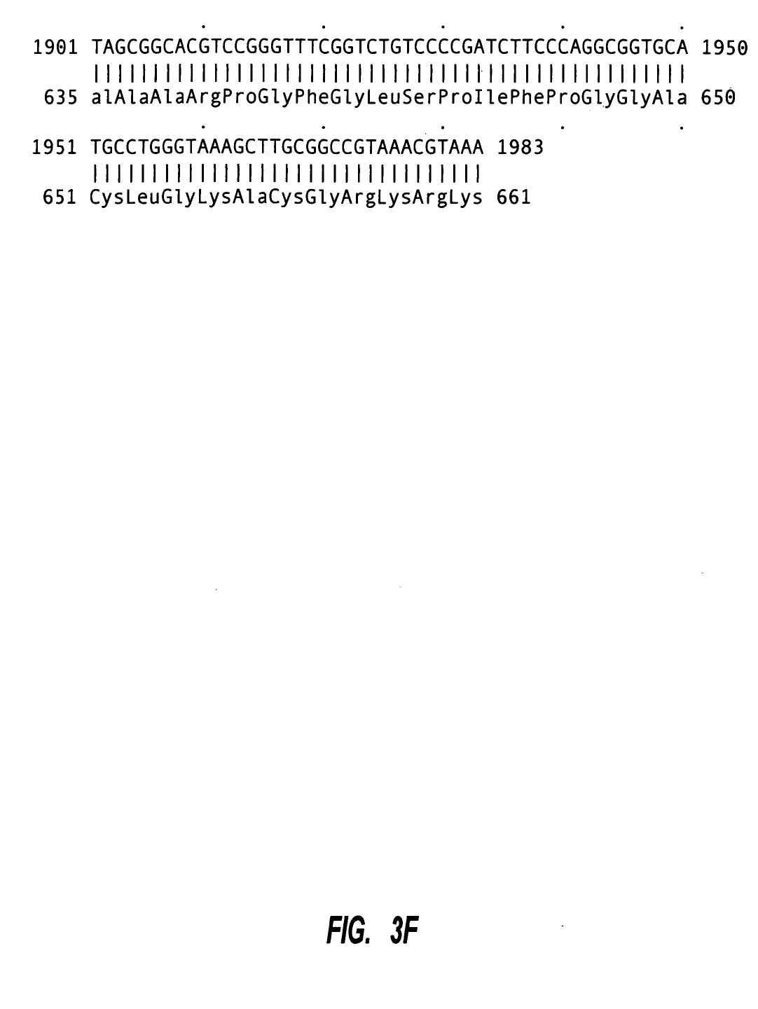

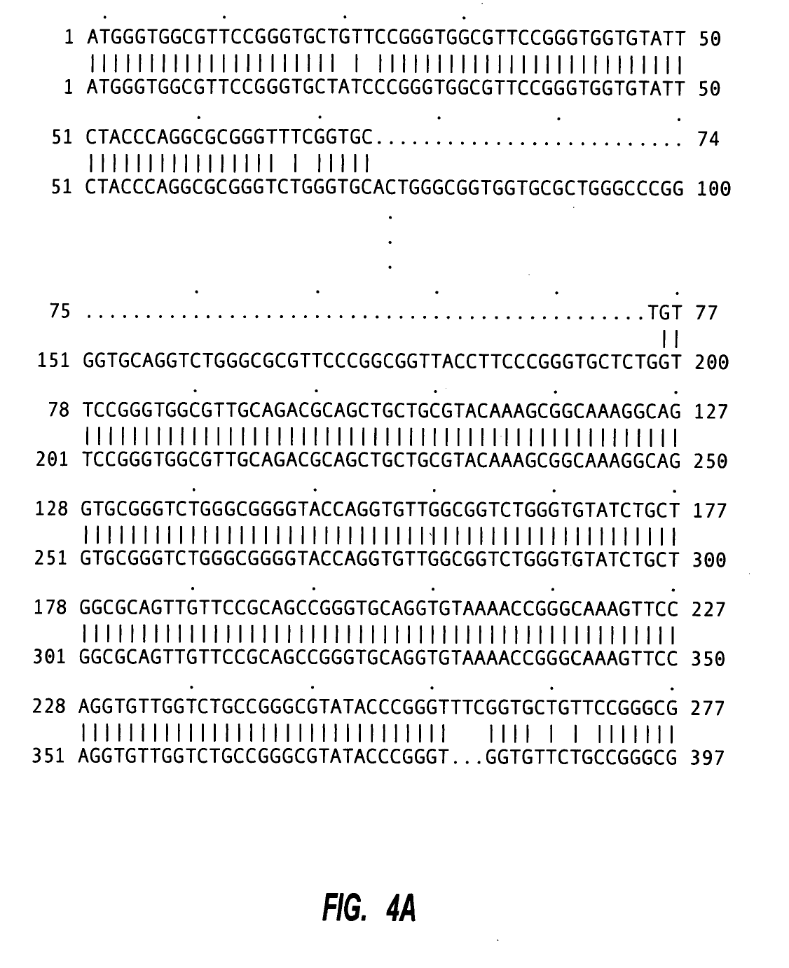

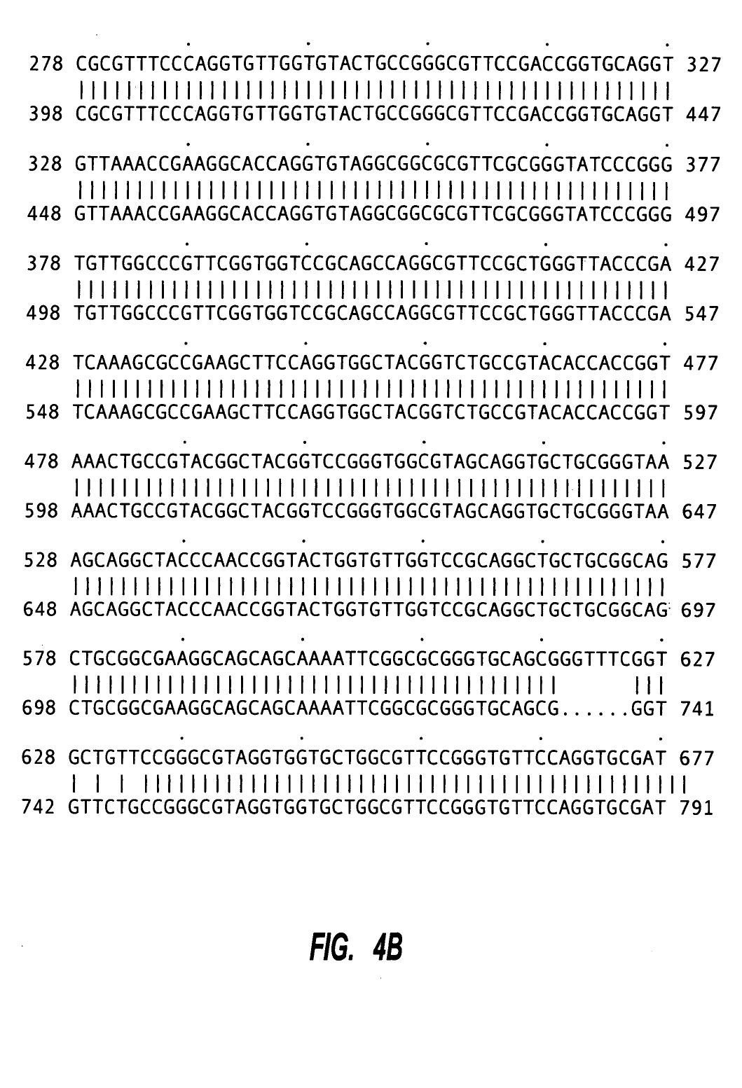

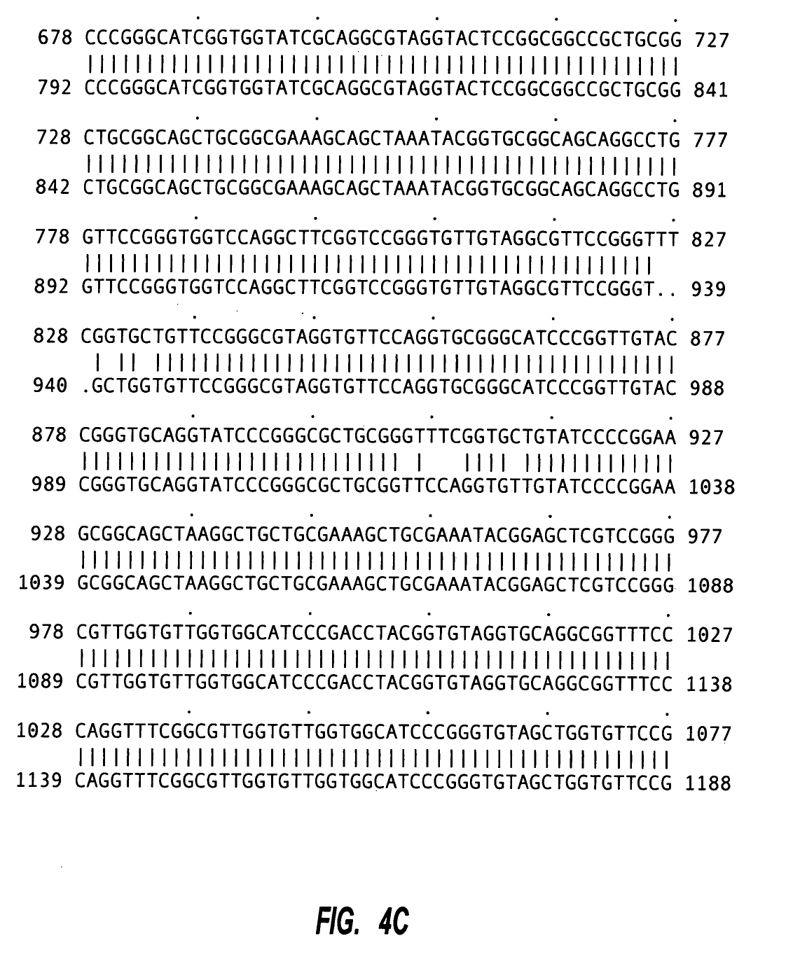

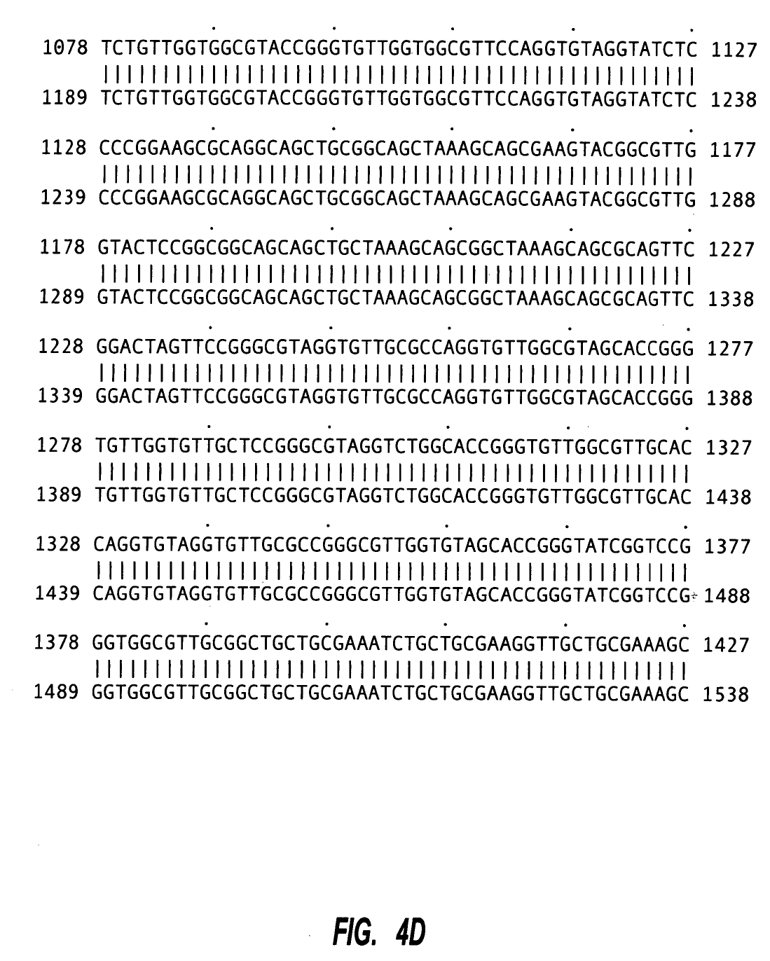

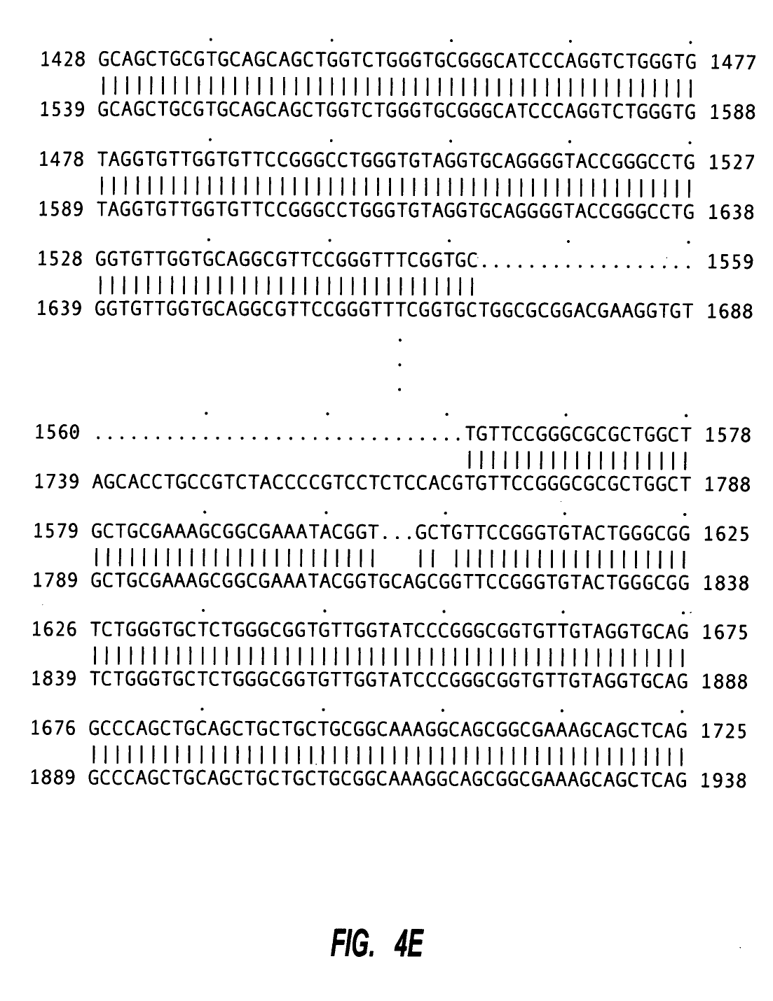

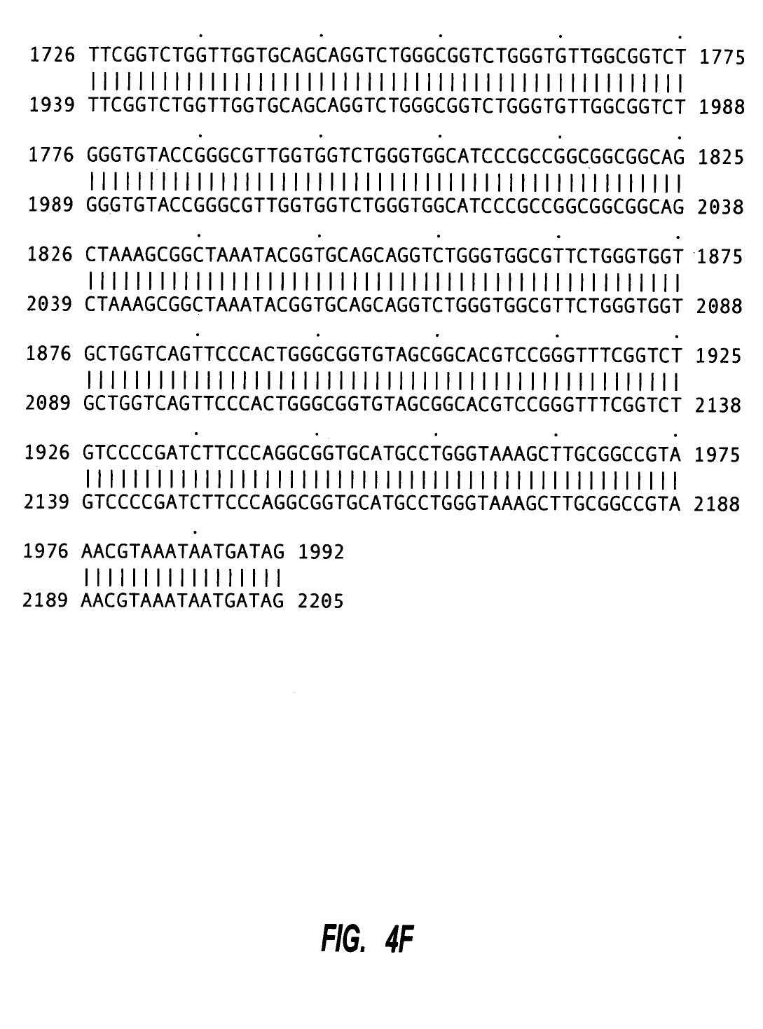

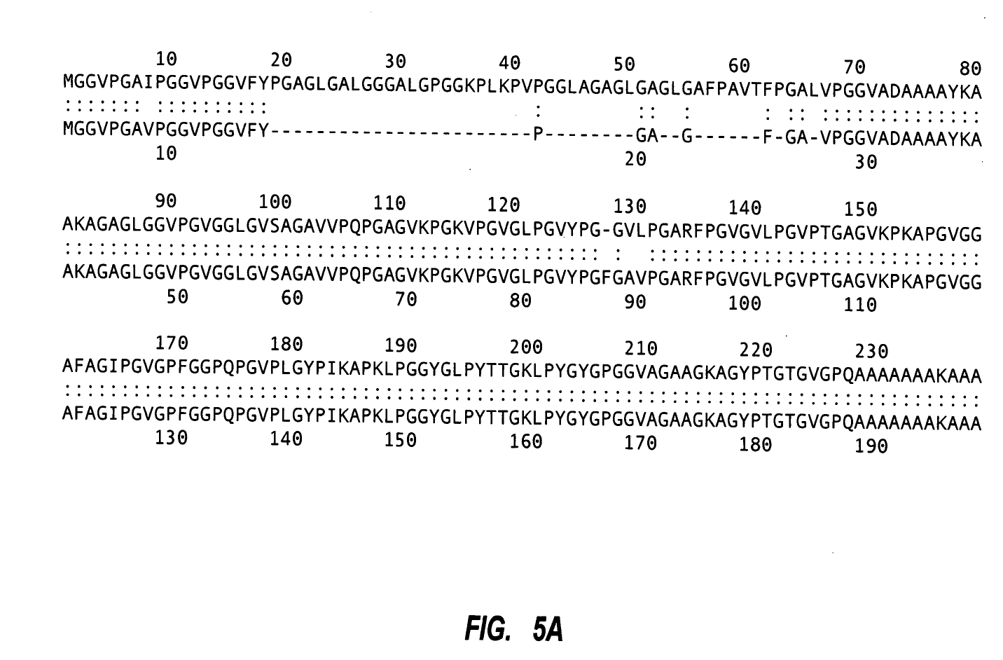

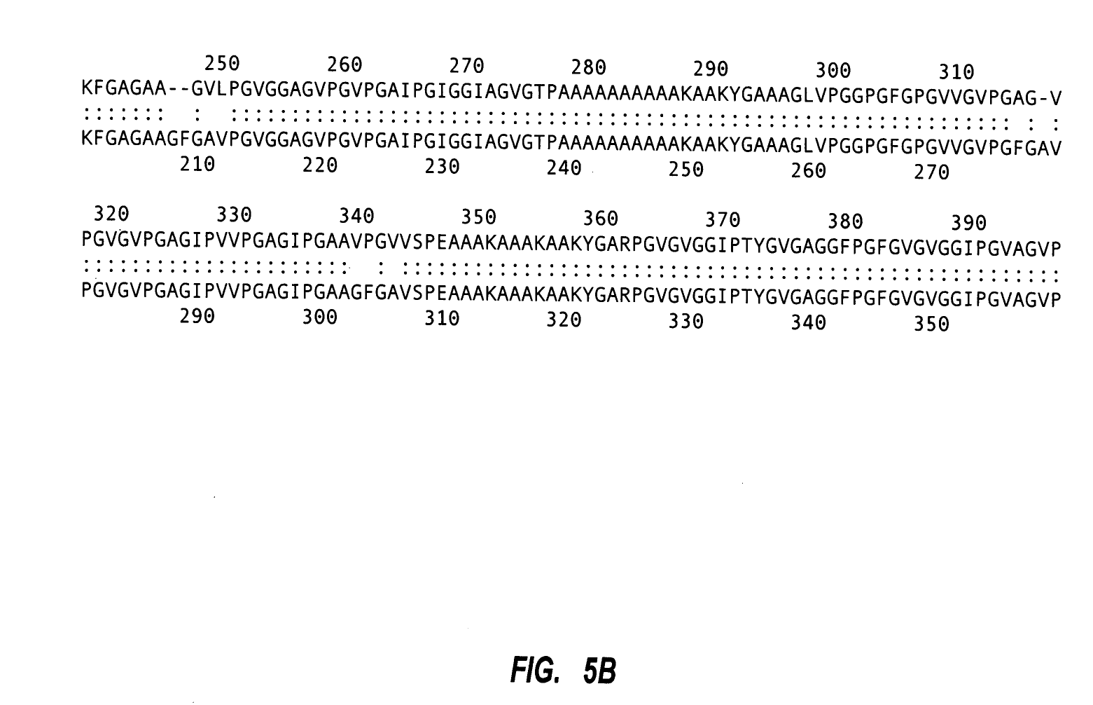

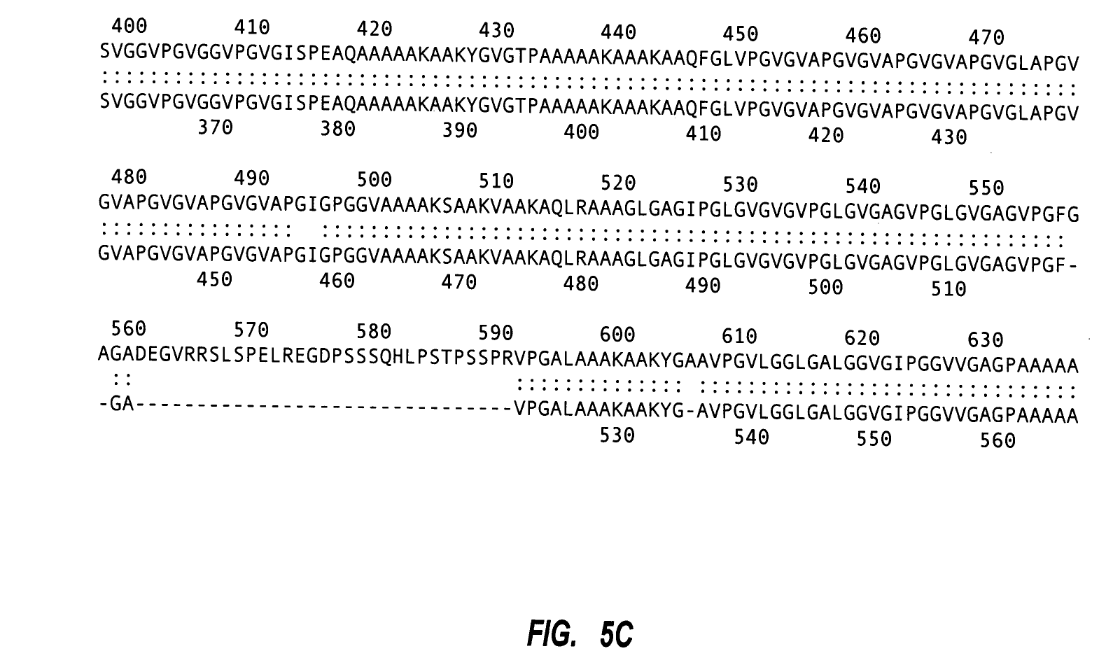

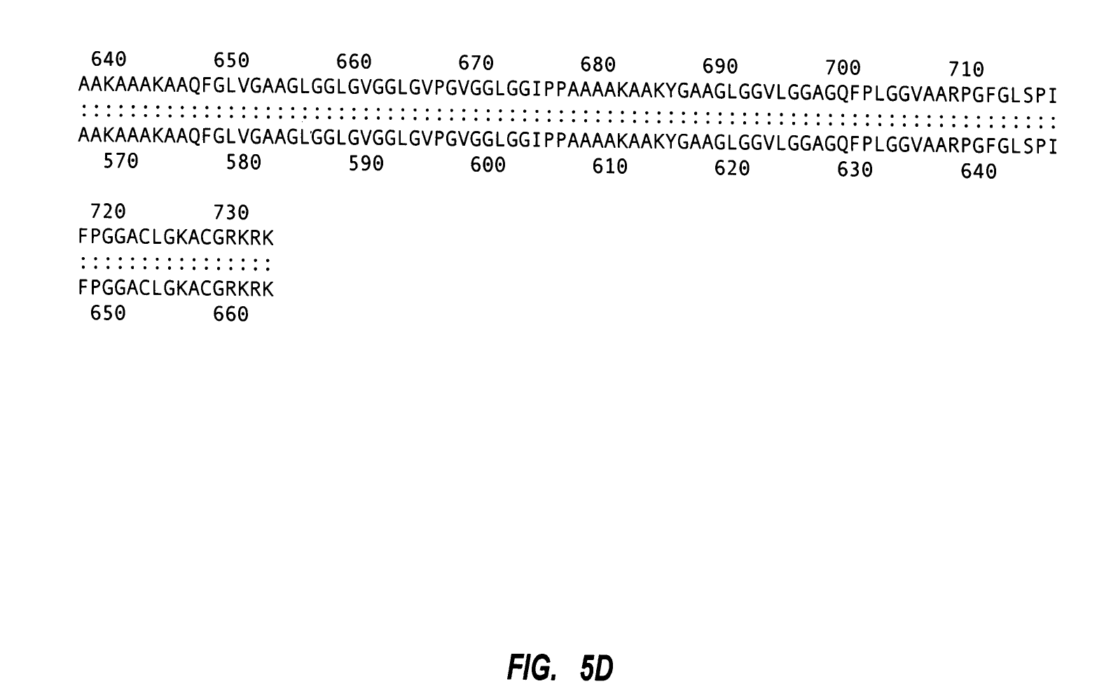

The preparation of SHEL is described in WO94/14958. It is directly expressed as a full length human protein with a calculated molecular weight of 64 kDa. The full nucleotide sequence and corresponding amino acid sequence of SHEL is shown in FIG. 1. The preparation of pSHELF is described in WO94/14958.

pSHELF differs from the natural coding sequence(s) in a number of ways. As described in WO94/14958, the untranslated regions present in the tropoelastin cDNA sequence were disregarded in designing the synthetic gene, and the nucleotides encoding the signal peptide were removed. Restriction endonuclease recognition sites were incorporated at regular intervals into the gene by typically altering only the third base of the relevant codons, thereby maintaining the primary sequence of the gene product. The facility for silent alteration of the coding sequence was also exploited to change the codon bias of the tropoelastin gene to that commonly found in highly expressed E. coli genes. [Genetics Computer Group (GCG) package version 7-UNIX using Codon Frequency and Gen Run Data: ecohigh-cod]. Two additional stop codons were added to the 3′-end, and an ATG start codon comprising a novel NcoI site was appended to the 5′-end. Bam HI cloning sites were engineered at both ends of the synthetic sequence. Since the gene contains no internal methionine residues, treatment of the newly-synthesized gene product (expressed directly or as a fusion with another gene) with cyanogen bromide would liberate a protein with the same or similar sequence as one form of natural tropoelastin comprising 731 amino acids. Other forms of processing are envisaged, which may generate tropoelastin species of the same or different lengths.

Two stop codons were added in order to allow the possible use of the construct in suppressor hosts, and also to avoid any potential depletion of termination (release) factors for translation As described in the following examples, the derivatives, pSHELFδ26A, pSHELFδ modified, pSHELgamma, pSHEL31-36, pSHEL32-36 and pSHELgammaδ26A were derived from the pSHELF nucleotide sequence. These particular derivatives, and indeed the derivaties, variants, expression products and hybrid molecules of the invention can equally be derived from a native human or non-human tropoelastin nucleotide sequence.

### EXAMPLE 1

### Construction of pSHELFδ26A and pSHELFδModified

Mutagenesis was used with PSHELF to remove DNA corresponding to exon 26A. The sequence of the mutagenic primer was:
5′CGG GTT TCG GTG CTG TTC CGG GCG CGC TGG 3′

This flanked either side of exon 26A by 15 bp resulting in its precise deletion. A second selection primer, which mutates a unique restriction site to another restriction site is normally used in the protocol but was not in this case since deletion of exon 26A also resulted in the deletion of a unique restriction site, PmlI. The enzyme PmlI was used to treat the mutation reaction to linearise any unmutated parental plasmid and consequently to enrich for mutant plasmid. The reaction mixture was used to transform competent BMH17-18 mutS E. coli, defective in mismatch repair, by electroporation and the entire transformed culture was grown overnight in LB+ampicillin. Mixed plasmid DNA, containing both mutated and parental plasmids, was isolated from the culture and the plasmid DNA was digested with PmlI to linearise the parental plasmid. The plasmid DNA, now enriched for mutated plasmid, was used to transform E. coli HMS174 by electroporation and transformants selected on LB plates containing 75 μgml⁻¹ ampicillin.

Colonies were grown overnight and plasmid mini-preparations performed. Constructs were screened using PmlI and those which were insensitive to digestion were further screened by KpnI/PstI double digestion. Candidate clones were sequenced to verify the sequence, named pSHELFδmodified.

Sequencing confirmed the region immediately surrounding the deletion was correct. PstI and BssHII restriction sites surrounding the correct region of pSHELFδmodified were used to remove the desired segment and re-insert it into the corresponding site of pSHELF. 6.5 μg PSHELF and 7.5 μg pSHELFδmodified were digested with BssHII, precipitated and digested with PstI. The appropriate three fragments were gel-purified and ligated. DNA was transformed into E. coli XL1-Blue and transformants selected on plates containing 75 μgml⁻¹ ampicillin.

Plasmids were isolated by mini-preparations and screened using BglI digestion. A candidate clone was further analysed by restriction enzyme digestion and sequenced, and named pSHELFδ26A.

### EXAMPLE 2

### Synthesis of Exon 26A

The region of SHEL corresponding to exon 26A was amplified by PCR, with primers modified to introduce an in-frame BamHII site upstream and a stop codon downstream at the 3′ end. Two forms were generated: one terminating in valine (26AV) and the other terminating in phenyalanine (26AF). These forms are as follows:
GADEGVRRSLSPELREGDPSSSQHLPSTPSSPRV with properties:
   Molecular weight=3588.80
   Residues=34
   Average Residue Weight=105.553
   Charge=−1
   Isoelectric point=5.71 and
GADEGVRRSLSPELREGDPSSSQHLPSTPSSPRF

A 26A coding region was expressed as a glutathione S-transferase (GST) fusion protein.

### EXAMPLE 3

### Glycosaminoqlycan Binding Activity of Exon 26A

Ultrafiltration assay methodology was developed to examine and quantify interactions occurring in vitro between the 26A region and purified extracellular matrix glcosaminoglycans. GST26A fusion protein and tropoelastin were compared with GST and tropoelastin lacking exon 26A at physiologicaly relevant conditions of pH and ionic strength.

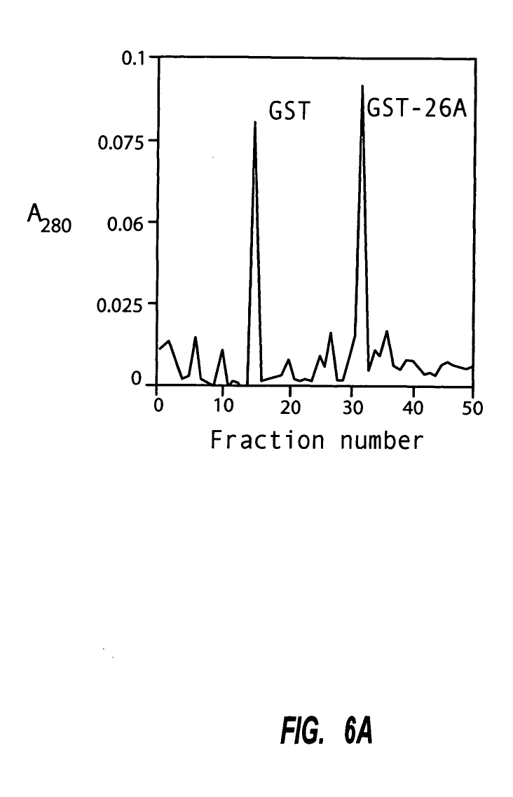

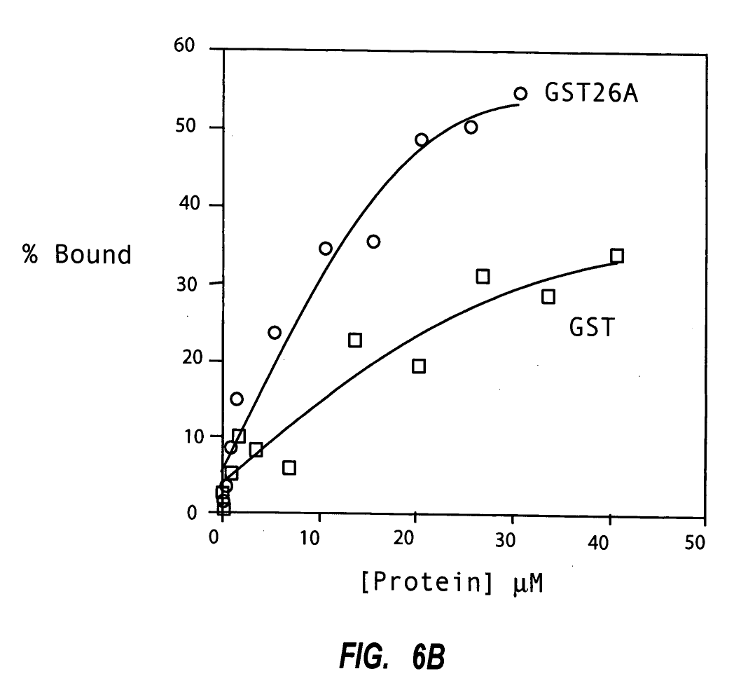

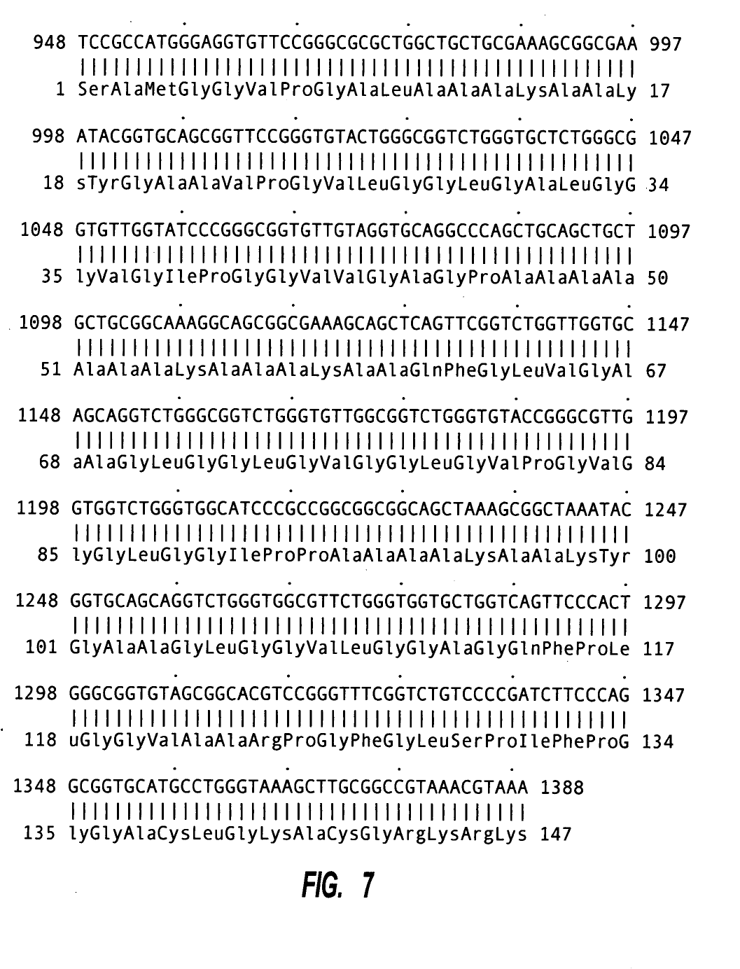

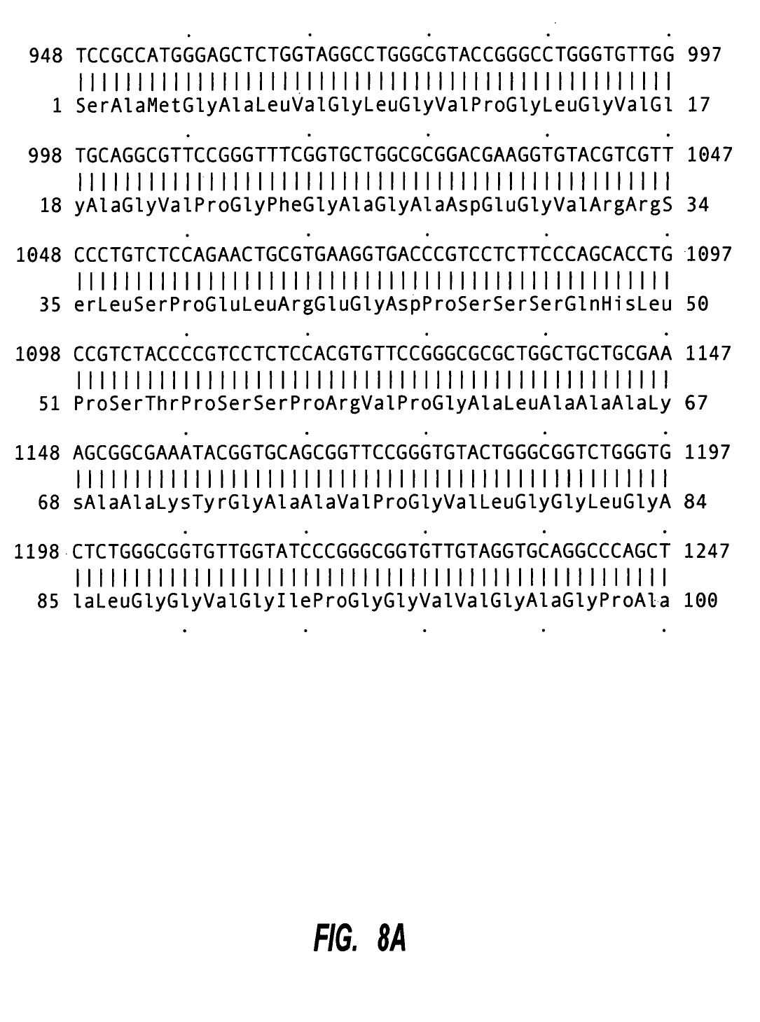

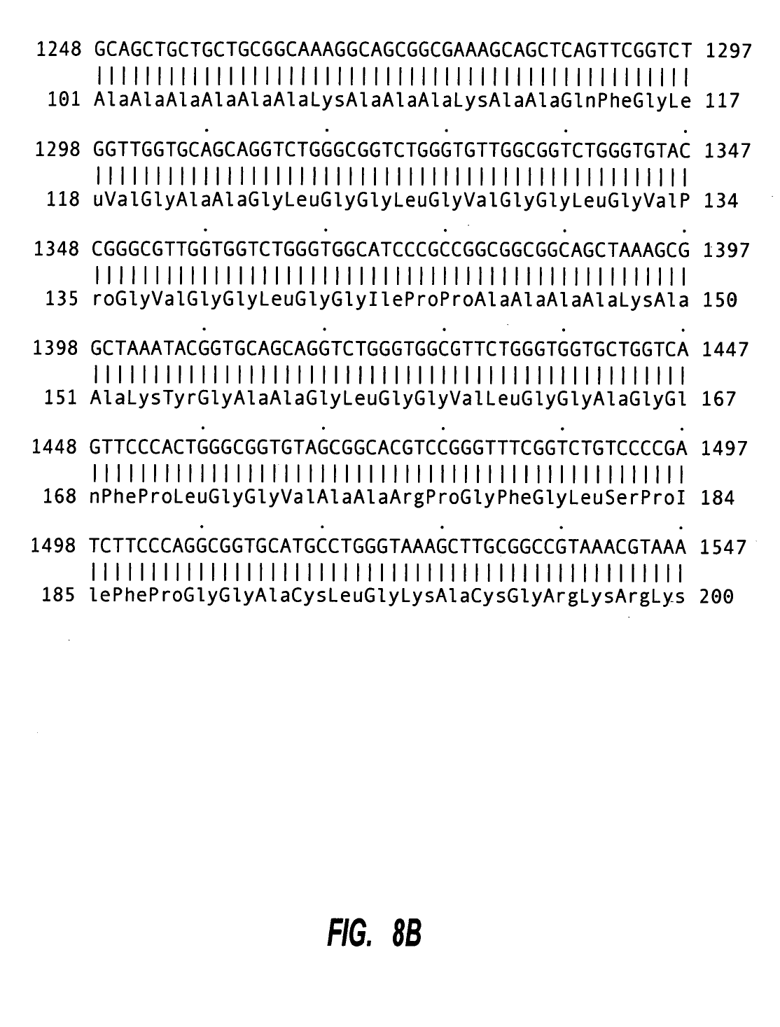

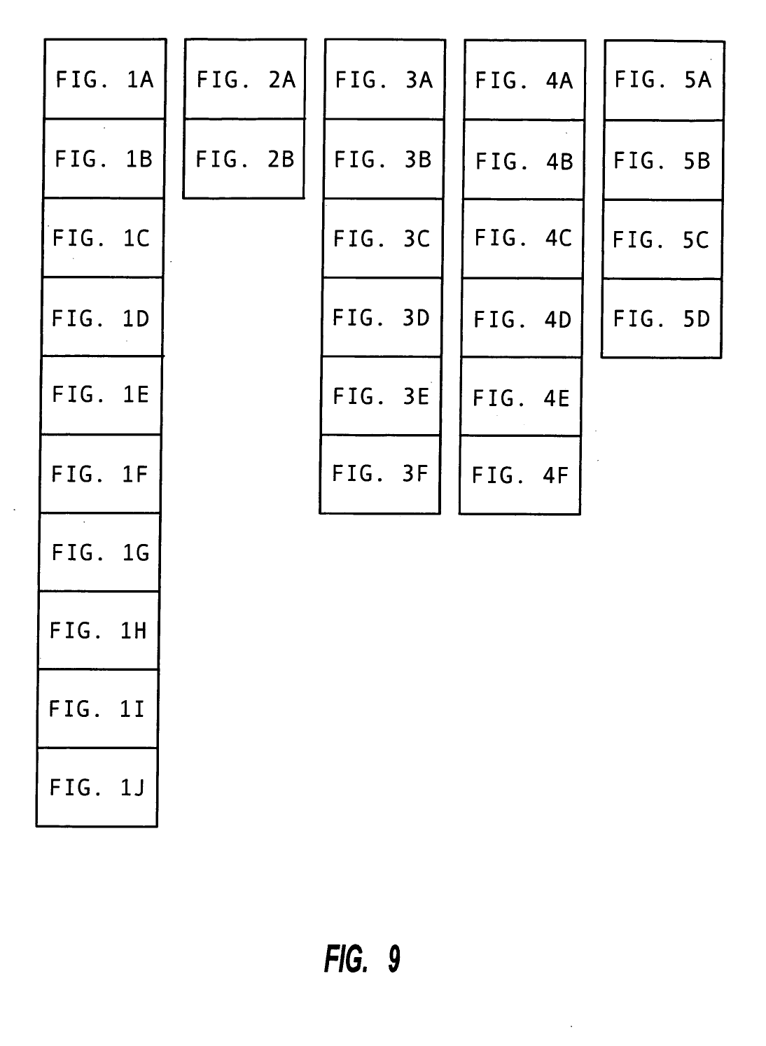

Experimental evidence supports the notion that peptide 26A (26AF and 26AV) binds GAGs. Immobilised heparin column binding shows that GST26A binds more tightly than does GST, and requires a higher sodium chloride concentration for elution (FIG. 6B). Furthermore, GST26A fusion protein binds radioactive heparin with greater efficiencies than GST, and these can be compared with GAGs including chondroitin sulphates and keratin sulphates. An implication of this is that GAGs binding to tropoelastin can be adjusted based upon the content of 26A. Cross-linked tropoelastin would be expected to show differential binding to GAGs based on the relative amounts of SHEL vs. SHELδ26A.

In summary, these studies reveal that the 26A region is a functional glycosaminoglycan binding domain, which functions in intact tropoelastin. It is also active when isolated as a fusion entity yet displays no detectable structure in the absence of bound GAG. Furthermore, panel competition studies indicate a preference for those GAGs found in close association with the elastic fibre in the extracellular matrix.

### EXAMPLE 4

### Construction of pSHELgamma, pSHEL31-36, pSHEL32-36 and pSHELgammaδ26A

pSHELgamma is derived from the pSHELgamma construct disclosed in WO94/14958. pSHEL31-36, pSHEL32-36 and pSHELgammaδ26A were derived from pSHELgamma. pSHELgamma was modified by introducing an oligonucleotide linker at the KpnI site. This encoded a factor Xa cleavage site which could be utilised in subsequent constructs. PCR and site directed mutagenesis was then used to generate further, shorter forms which provided fusions with GST. Constructs were DNA sequenced for verification. Induced protein was isolated as GST-fusion proteins, which were subsequently bound to glutathione agarose. Protease cleavage was optional where fusion proteins were desired; otherwise the cleaved proteins and peptides were further purified by reverse phase HPLC.

### INDUSTRIAL APPLICATION

The derivatives and expression products of the invention are of use in inter alia the medical, pharmaceutical, veterinary and cosmetic fields.

### REFERENCES

1. Indik Z, Yeh H, Ornstein-Goldstein N, Sheppard P, Anderson N, Rosenbloom J C, Peltonen L and Rosenbloom J (1987) PNAS (USA) 84 5680-5684
2. Indik Z, Abrams W. R., Kucich U, Gibson C. W., Mecham R. P. and Rosenbloom J (1990) Arch. Biochem Biophys 280 80-86
3. Oliver L, Luvalle P A, Davidson J. M., Rosenbloom J, Mathew C. G., Betser A. J. and Boyd C. D. (1987) Collagen Rel Res 7 77-89
4. Sambrook J., Fritsch E. F., and Maniatis T. (1989) Molecular cloning: a laboratory manual, second edition Cold Spring Harbor Laboratory Press, Cold Spring Harbor, N.Y.
5. Bressan G. M., Argos P. and Stanley K. K. (1987) Biochemistry 26 1497-11503
6. Raju K. aand Anwar R. A. (1987) J. Biol Chem 262 5755-5762
7. Pierce R. A., Alatawi A, Deak S. B. & Boyd C. D. (1992) Genomics 12 651-658
8. Lipman and Pearson (1985) Science 227, 1435.
9. Bedell-Hogan, D., Trackman, P., Abrams, W., Rosenbloom, J. and Kagan H. (1993) J. Biol. Chem. 268, 10345-10350
10. Studier, F. W., Rosenberg, A. H., Dunn, J. J. and Dubendorff, J. W. (1990) Methods Enzymol. 185, 60-89
11. Gough, J., and Murray, N. (1983) J. Mol. Biol. 166, 1-19
12. Bullock, W. O., Fernandez, J. M. and Short, J. M. (1987) BioTechniques 5, 376-379
13. Slack, J. L., Liska, D. J. and Bornstein P. (1991) Mol. Cell Biol. 11: 2066-2074
14. Janne, J., Hyttinen, J. M., Peura, T., Tolvanen, M., Alhonen, L. And Halmekyto M. (1992) Ann. Med. 24: 273-280.
15. Merrifield, R. B., (1963) J. Am. Chem. Soc. 85: 2149-2154.
16. Knorr R., Trzeciak, Bannarth W., Gillessen, D. (1989) Tetrahedron Letters 30: 1927-1930

1-81. (canceled)

82. A method of imparting glycosaminoglycan binding activity to a polymer or enhancing the glycosaminoglycan binding activity of a polymer, comprising the step of linking to the polymer a tropoelastin derivative consisting of an amino acid sequence selected from the group consisting of SEQ ID NO: 12 and SEQ ID NO: 13.

83. A method as in claim 82 wherein the polymer is a biological polymer.

84. A method as in claim 83 wherein the biological polymer is selected from the group consisting of protein, lipid, sugar and nucleic acid.

85. A method as in claim 83 wherein the biological polymer is a protein.

86. A method as in claim 85 wherein the protein is selected from the group consisting of growth factors, cytokines and antibodies.

87. A hybrid molecule produced by the method of claim 82.

88. A hybrid molecule as in claim 87 wherein the polymer is a biological polymer selected from the group consisting of protein, lipid, sugar and nucleic acid.

89. A hybrid molecule as in claim 88 wherein the biological polymer is a protein.

90. A hybrid molecule as in claim 89 wherein the protein is selected from the group consisting of growth factors, cytokines and antibodies.

91. A hybrid molecule as in wherein the polymer is a synthetic polymer.

92. A hybrid molecule comprising a polymer linked to a tropoelastin derivative, wherein the tropoelastin derivative comprises of an amino acid sequence selected from the group consisting of SEQ ID NO: 12 and SEQ ID NO: 13.

93. A hybrid molecule as in claim 92 wherein the polymer is a biological polymer.

94. A hybrid molecule as in claim 93 wherein the biological polymer is selected from the group consisting of protein, lipid, sugar and nucleic acid.

95. A hybrid molecule as in claim 94 wherein the biological polymer is a protein.

96. A hybrid molecule as in claim 95 wherein the protein is selected from the group consisting of growth factors, cytokines and antibodies.

97. A hybrid molecule as in claim 92 wherein the polymer is a synthetic polymer.

98. An isolated tropoelastin derivative comprising an amino acid sequence selected from the group consisting of SEQ ID NO: 12 and SEQ ID NO: 13.

99. A tropoelastin derivative as in claim 98 comprising an amino acid sequence selected from the group consisting of SEQ ID NO: 9 and SEQ ID NO: 14.

100. A polynucleotide encoding the tropoelastin derivative of claim 98.

101. A polynucleotide as in claim 100 comprising the nucleotide sequence of SEQ ID NO: 8.

102. A polynucleotide as in claim 100 wherein the polynucleotide is a synthetic polynucleotide.

103. An isolated tropoelastin derivative produced by a method including the following steps:
(a) introducing a polynucleotide for encoding a derivative according to claim 98 into a cell;
(b) maintaining the cell under conditions to allow expression of the polynucleotide to produce the derivative; and
(c) isolating the derivative.

104. A hybrid molecule as in claim 87 wherein the hybrid molecule is contained in a formulation further comprising a pharmaceutically acceptable carrier or diluent.

105. A tropoelastin derivative as in claim 98 wherein the tropoelastin derivative is contained in a formula comprising a pharmaceutically acceptable carrier or diluent.

106. A tropoelastin derivative as in claim 98 wherein the tropoelastin derivative is contained in an expression product.

107. A tropoelastin derivative contained in an expression product as in claim 106 wherein said tropoelastin derivative consists of an amino acid sequence selected from the group consisting of SEQ ID NO: 12 and SEQ ID NO: 13.

108. A tropoelastin derivative as in claim 98 which is recombinant.
